(19) United States
(12) Patent Application Publication
(10) Pub. No.: US 2025/0058109 A1
(43) Pub. Date: Feb. 20, 2025

(54) MIXED DIRECT MACRO-CURRENT ELECTROTHERAPY SYSTEM FOR TARGETED TREATMENT OF PHYSICAL CONDITION AND AFFECTED TISSUE

(71) Applicant: Neurological Fitness Equipment and Education LLC, Austin, TX (US)

(72) Inventors: Garrett Salpeter, Austin, TX (US); Erik Leander, Boca Raton, FL (US)

(73) Assignee: Neurological Fitness Equipment and Education LLC, Austin, TX (US)

(21) Appl. No.: 18/234,214

(22) Filed: Aug. 15, 2023

(51) Int. Cl. A61N 1/36; A41D 19/00; A61H 99/00; A61N 1/04

(52) U.S. Cl. CPC A61N 1/36034; A41D 19/0024; A61H 99/00; A61N 1/0452; A61N 1/0472; A61N 1/36031; A61H 2201/10

(57) ABSTRACT

A mixed direct macro-current (MDMC) electrotherapy system may comprise an MDMC electrotherapy pulsed frequency generator to deliver, during treatment of an identified condition affecting an identified tissue type, identified based on the practitioner's observance of or the patient's reporting of the rhythmic contraction of the patient's muscle, a first MDMC pulsed current frequency having a tissue or condition treatment pulse frequency corresponding to the identified tissue type to the patient's skin including an alternating current (AC) current frequency component, with the first MDMC pulsed frequencies causing rhythmic contraction of the patient's muscle within a physical manipulation target region at a frequency below the tissue or condition treatment pulse frequency.

### FIELD OF THE DISCLOSURE

The present disclosure generally relates to electrotherapy techniques for treatment of a patient's tissues. More specifically, the present disclosure relates to electrotherapy applying to a physical manipulation target region of a patient's body a first applied current frequency targeted toward treatment of a patient's identified condition and a second applied current frequency targeted toward treatment of a patient's tissues affected thereby.

### BACKGROUND

Application of frequency-specific micro-current (e.g., less than one milliampere) applied current frequencies may be applied to various tissues in patients' bodies to reduce inflammation of targeted tissues. Further, certain micro-current frequencies may be used to treat known or identified physical conditions, including but not limited to inflammation, tears, fractures, or more. Alternating current (AC) applied current frequencies or direct current (DC) electrical pulses having a current under one mA (e.g., “micro-current”) may have an advantage for patient comfort over DC electrical pulses having a current above one mA (e.g., direct “macro-current” or “DMC”) that have been associated with prickling or painful sensation in the patient's skin during electrotherapy treatment.

### BRIEF DESCRIPTION OF THE DRAWINGS

It will be appreciated that for simplicity and clarity of illustration, elements illustrated in the Figures are not necessarily drawn to scale. For example, the dimensions of some elements may be exaggerated relative to other elements. Embodiments incorporating teachings of the present disclosure are shown and described with respect to the drawings herein, in which:

FIG. 1 is a graphical diagram illustrating a mixed direct macro-current (MDMC) electrotherapy system according to an embodiment of the present disclosure;

FIG. 2 is a graphical diagram illustrating an MDMC electrotherapy pulsed frequency generator according to an embodiment of the present disclosure;

FIG. 3 is a graphical diagram illustrating internal components of an MDMC electrotherapy pulsed frequency generator according to another embodiment of the present disclosure;

FIG. 4 is a graphical diagram illustrating MDMC pulsed frequencies according to an embodiment of the present disclosure;

FIG. 5 is a flow diagram illustrating a method of determining a tissue treatment pulse frequency and a condition treatment pulse frequency for treatment of an identified tissue condition according to an embodiment of the present disclosure;

FIG. 6 is a flow diagram illustrating a method of diagnosing a patient's physical condition and tissue for treatment according to an embodiment of the present disclosure; and

FIG. 7 is a flow diagram illustrating a method of treating a patient's tissue affected by an identified condition according to an embodiment of the present disclosure.

The use of the same reference symbols in different drawings may indicate similar or identical items.

### DETAILED DESCRIPTION OF THE DRAWINGS

The following description in combination with the Figures is provided to assist in understanding the teachings disclosed herein. The description is focused on specific implementations and embodiments of the teachings, and is provided to assist in describing the teachings. This focus should not be interpreted as a limitation on the scope or applicability of the teachings.

Frequency-specific micro-current (e.g., having a current of less than one milliampere (mA)) electrotherapy has been used to treat known conditions affecting specific tissues that may be causing a patient's reported physical symptoms. Existing electrotherapy systems apply an electrical current to a patient's musculature or soft tissue via one or more electrodes or similar structures placed in contact with a patient's skin. Frequency-specific electrotherapy involves the application of either an alternating current (AC) applied current frequencies having a frequency known to treat a specific condition or tissue type, or a direct current (DC) electrical pulse applied at the same frequency. AC applied current frequencies applied to a patient's skin may inhibit ionization of skin or adipose tissues known to cause a prickling or painful sensation. Embodiments of the present disclosure apply applied current frequencies comprised of a DC electrical pulse mixed with an AC applied current frequency such that the AC applied current frequency inhibits any prickling or painful sensation that may otherwise be caused by application of the DC electrical pulse.

The DC electrical pulse applied in such a way in various embodiments of the present disclosure may have a magnitude of current above one milliampere (mA), which may be referred to herein as direct “macro-current” (e.g., “DMC”). Reference to such DMC electrical pulses may distinguish these electrical pulses from direct micro-current electrical pulses having a magnitude of current below one mA, as used in previous systems. In such previous systems, the magnitude of current for direct micro-current electrical pulses may be capped below one mA specifically to avoid the prickling or painful sensation associated with DMC electrical pulses applied in isolation (e.g., without the mixed AC wave described directly above) to a patient's skin. As described directly above and throughout this disclosure, mixing a DMC electrical pulse with an AC electromagnetic wave, for a pulsed mixed direct macrocurrent (MDMC) EM wave may increase the range of current magnitudes (e.g., above one mA) that may be employed while still avoiding the prickling or painful sensations associated with higher current magnitudes.

Microcurrent electrical pulses employed in existing systems produce no effect visible to the practitioner or observable by the patient during the electrotherapy session to indicate whether the proper frequencies are being applied. For example, the current of the electricity so applied may be too low. Many identified conditions and tissue types are known in existing systems to respond to electrotherapy treatment at more than one frequency. Each patient's individual condition and tissue type may respond best to one of several known treatment pulse frequencies. This may result in practitioners testing a variety of known frequencies before isolating the optimal frequency for treatment of each individual patient's condition or tissue type. Systems that employ microcurrent electrical pulses may require multiple treatment sessions in order to isolate a frequency targeted to a patient's physical condition, and a further series of treatment sessions to isolate a frequency targeted to a specific tissue type affected thereby. Thus, patients may undergo one or more sessions in which an ineffective or non-optimal frequency is applied, and limited beneficial effects are produced, unnecessarily increasing cost of treatment and lengthening time required for healing.

Previous frequency-specific electrotherapy systems also fail to simultaneously treat both a physical condition responsive to a particular condition treatment pulse frequency and a tissue type affected by that condition and responsive to a separate tissue treatment pulse frequency. A system that allows a practitioner to isolate an effective or optimal frequency for treatment of the patient's physical condition and an effective or optimal frequency for treatment of a tissue type affected thereby during a single electrotherapy session is needed.

The mixed direct macro-current (MDMC) electrotherapy system in embodiments of the present disclosure addresses these issues by simultaneously applying to a patient's physical manipulation target region two MDMC pulsed frequencies comprising a mixture of a DC electrical pulse applied at a given pulse frequency and an AC applied current frequency. In embodiments described herein, a first MDMC pulsed current frequency with a first pulse frequency targeted toward treatment of an identified condition, and a second MDMC pulsed current frequency with a second pulse frequency targeted toward treatment of a tissue affected by the identified condition may be applied to the patient's physical manipulation target region. Because each of the first and second MDMC pulsed frequencies may have a magnitude of current above one mA, the combined application of these first and second MDMC pulsed frequencies may produce visible or noticeable effects during the electrotherapy session. For example, application of these MDMC pulsed frequencies may produce contraction or relaxation of the tissues within the patient's physical manipulation target region.

In some cases, application of these MDMC pulsed frequencies may cause rhythmic contractions of the muscle nearby tissues targeted for treatment at a rate that does not match or correlate to either of the MDMC pulsed frequencies being applied. In some cases, the tissue targeted for treatment may be the muscle contracting, and in other cases, the muscle contracting may be situated nearby the targeted tissue, such as bone, cartilage, fascia, etc. For example, application of first and second MDMC pulsed frequencies having pulse frequencies (e.g., DMC pulsed at a specific frequency that is mixed with the AC applied current frequency) above 10 Hz may produce an observable rhythmic contraction of the patient's muscles within the physical manipulation target region at a frequency below 5 Hz. This may occur even if the tissue targeted for treatment is a tissue other than the muscle contracting (e.g., nearby bone, cartilage, ligament, etc.).

Because the frequency of these contractions may fall well below the pulse frequencies of the MDMC pulsed frequencies, one may conclude that the rhythmic contraction of the patient's muscles is not directly caused by the application of these MDMC pulsed frequencies. Rather, the rhythmic contraction of the patient's muscles indicates a response by the tissues targeted for treatment to the application of these MDMC pulsed frequencies. A patient's muscles nearby targeted tissues tend to produce these rhythmic contractions upon application of MDMC pulsed frequencies having MDMC pulse frequencies similar to those systems employing direct microcurrent electrical pulses, but the current embodiments of the present disclosure utilize a sufficient current to elicit the rhythmic contraction response for muscle within the treatment area) to target healing of the specific tissues nearby muscles undergoing these contractions, or to target a specific condition affecting the tissues nearby muscles undergoing these contractions. In other words, use of MDMC pulsed frequencies provides real-time and observable feedback practitioners may use to isolate frequencies of applied MDMC pulsed frequencies to which the patient's tissues respond positively. The present disclosure thus describes a solution that allows a practitioner to determine the optimal frequency for treating a patient's condition and the optimal frequency for treating a patient's tissues affected thereby during a single session, reducing cost and overall treatment time.

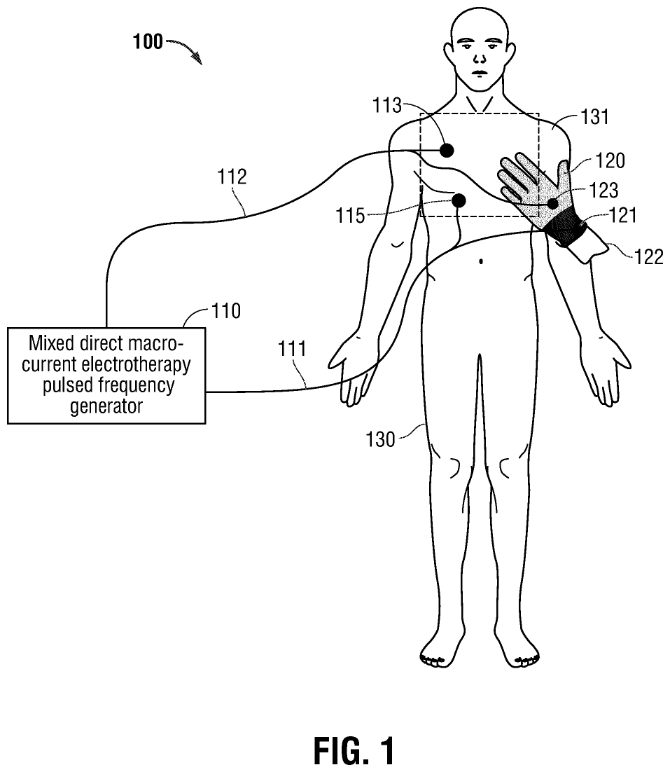

FIG. 1 is a graphical diagram illustrating a mixed direct macro-current (MDMC) electrotherapy system for treatment of an identified condition affecting a specific tissue type of a patient according to an embodiment of the present disclosure. As described herein, the mixed direct macro-current (MDMC) electrotherapy system 100 in an embodiment may simultaneously apply to a patient's 130 physical manipulation target region 131 one or more separate mixed direct macro-current (MDMC) MDMC pulsed frequencies and each or both mixed with an AC current frequency generated by the mixed direct macro-current (MDMC) electrotherapy frequency generator 110. A physical manipulation treatment session in an embodiment may begin with the patient 130 identifying an area of complaint in which a previously identified condition is affecting a known tissue of the patient's body 130, or in which an undiagnosed condition is affecting a plurality of possible tissue types within the area of complaint or within a nearby area of the patient's body 130. The practitioner may identify, based on this information, a physical manipulation target region 131 in which the practitioner may apply electrotherapy techniques or physical manipulation treatment of the patient's tissues in order to treat the condition and tissue type underlying the patient's 130 complaints.

In some embodiments, the practitioner may determine that the physical manipulation target region 131 coincides, overlaps, or identically matches the patient's area of complaint. For example, a patient may complain of pain in the chest region that the practitioner determines is being caused by some condition of one of the tissue types within that same region of the patient's 130 chest, such as pectoral muscles or associated tendons or ligaments. In such an embodiment, the practitioner may determine that the condition or tissue in need of treatment to address the patient-identified pain is within a physical manipulation target region 131 including the patient's 130 chest. In other embodiments, the practitioner may determine that the patient's complaints indicate referred pain being caused by a condition or tissue lying outside the patient's area of complaint. For example, a patient may complain of pain in the upper thighs that the practitioner identifies as being caused by an injury to the patient's lower back. In such an example embodiment, the patient's area of complaint may include the upper thighs (e.g., quadriceps), but the practitioner may determine that the condition or tissue in need of treatment to address this pain is within a physical manipulation target region including the patient's 130 lower back.

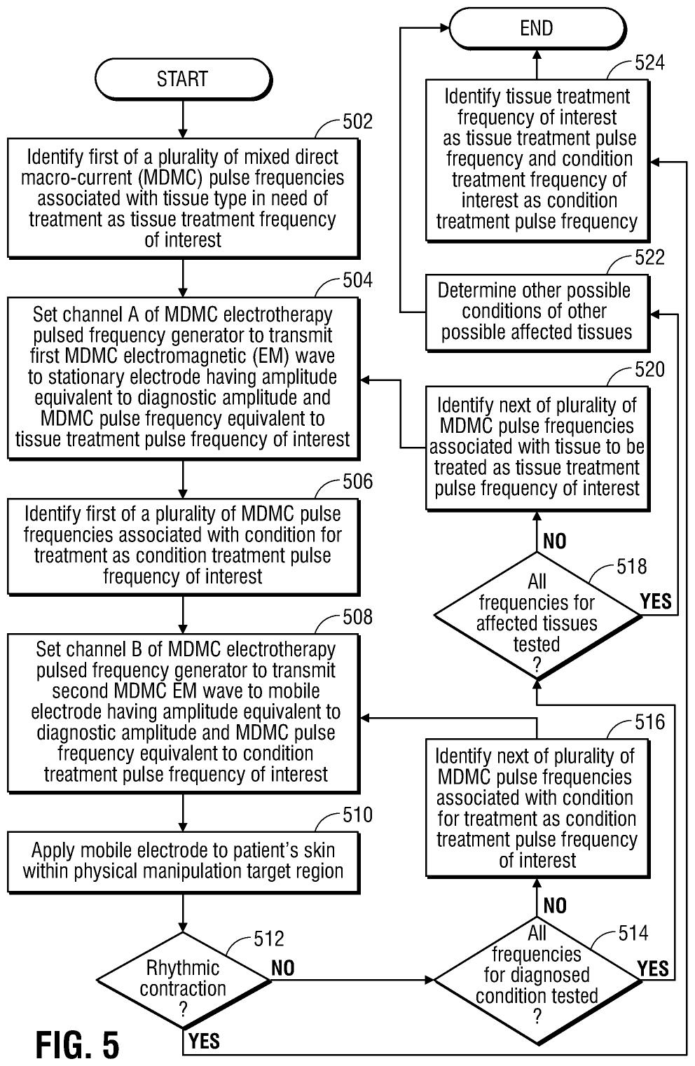

As described herein, a practitioner may apply one or more separate mixed direct macro-current (MDMC) applied current frequencies generated by the MDMC electrotherapy pulsed frequency generator 110 to treat a tissue type and a condition affecting tissues within the physical manipulation target region 131 in an embodiment. These waves may include, for example, a first MDMC pulsed current frequency with a first pulse frequency targeted toward treatment of the patient's 130 tissue types, and a second MDMC pulsed current frequency with a second pulse frequency targeted toward treatment of a condition affecting the patient's 130 tissue, as discussed in greater detail below with respect to FIGS. 5, 6 and 7 below. In other embodiments, such first and second MDMC pulsed frequencies may be applied to the patient's 130 physical manipulation target region 131, in which the patient 130 is experiencing pain or discomfort caused by an unknown or undiagnosed condition impacting potentially several different tissue types in order to isolate or diagnose the condition and tissue affected thereby within the physical manipulation target region 131, as discussed in greater detail below.

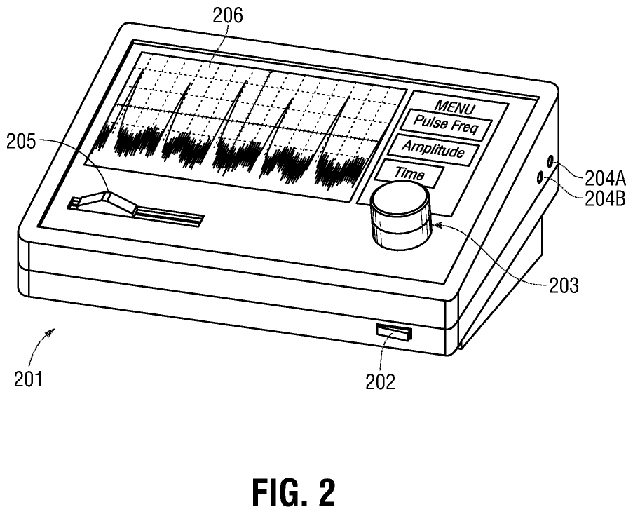

A first or second MDMC pulsed current frequency in an embodiment may generated by the MDMC electrotherapy pulsed frequency generator 110 according to instructions or input provided by a practitioner 122, as described in greater detail with respect to FIG. 2, below (e.g., setting an amplitude or an MDMC pulse frequency for an MDMC pulsed current frequency, based on a known or suspected condition affecting a known or suspected tissue of the patient 130). As described herein, each MDMC pulsed current frequency may comprise an Alternating Current (AC) electromagnetic (EM) wave mixed with Direct MacroCurrent (DMC) electrical pulses applied at a practitioner-selected frequency. The practitioner-selected frequency for the DMC electrical pulses within each MDMC pulsed current frequency may represent the MDMC pulse frequency known to treat a specific tissue type or condition, as described herein.

A first MDMC pulsed current frequency may be transmitted from the MDMC electrotherapy pulsed frequency generator 110 in an embodiment via an electrically conductive wire 112 to an electrode pair affixed to or in contact with the patient's 130 skin. For example, a stationary electrode 113 may be affixed to the patient's 130 skin within the physical manipulation target region 131 and a mobile electrode 123 may be applied to a user's skin via an electrotherapy glove 120. In other embodiments, the stationary electrode 113 may be placed anywhere on the patient's body in which the electrode may come into direct contact with the patient's skin and provide a loop via line 112 to the MDMC electrotherapy pulsed frequency generator 110 for applying a first MDMC pulsed frequency with AC component. In yet another embodiment, plural stationary electrodes 113 and 115 may be used. In another embodiment, a second stationary ground electrode 115 may also applied to the patient's skin within the physical manipulation target region 131 (or anywhere else on the patient's body) and be paired with a second mobile electrode 121 of the electrotherapy glove 120 which may receive electrical current, causing the first MDMC pulsed frequency applied with an AC component via the mobile electrode 123 or 121 to travel through the patient's tissues within the physical manipulation target region 131, toward the paired stationary ground electrode 113 or 115 respectively, and return to the MDMC electrotherapy pulsed frequency generator 110 via line 112 or 111. The stationary electrodes 113 and 115 in an embodiment may be referred to herein as “stationary” because the practitioner does not move this electrode with respect to the patient's body 130 during the physical manipulation or electrotherapy treatment session as compared to mobile electrodes 123 and 121 deployed on an electrotherapy glove 120 or other tool used by a practitioner to provide electrotherapy, physical manipulation therapy, or both according to embodiments herein. Such a mobile electrodes 121 or 123, in an embodiment, may be referred to herein as “mobile” because it may be moved across the patient's 130 skin within the physical manipulation target region 131 by the practitioner 122 during an electrotherapy-assisted physical manipulation treatment. In some embodiments, the mobile electrode 120 may comprise an electrotherapy glove worn by a practitioner 122.

In an embodiment, the mixed direct macro-current (MDMC) electrotherapy pulsed frequency generator 110 may also transmit a second MDMC pulsed frequency to a mobile electrode 121 of an electrotherapy glove 120 operated by a practitioner. A static electrode 115, paired with mobile electrode 121 may receive electrical current, causing the second MDMC pulsed current frequency applied via the mobile electrode 121 to travel through the patient's tissues within the physical manipulation target region 131, toward the static electrode 115, and return to the MDMC electrotherapy pulsed frequency generator 110 via line 111. Similarly, paired electrodes 123 and 113 may be used to provide the second MDMC pulsed frequency. An electrotherapy glove (e.g., 120) in an embodiment may deliver the second MDMC pulsed current frequency through one or more electrode pairs situated at various locations along the exterior surface of the electrotherapy glove, or through electrically conductive fibers interwoven within the exterior surface of the electrotherapy glove. In other embodiments, the mobile electrodes 121 or 123 may comprise an electrode affixed to a hand-held tool or implement for manipulating the patient's 130 tissues through applied pressure (e.g., massage, chiropractic, or manual physical therapies). The mobile electrode 123 paired with static electrode 113 in an embodiment may receive the second MDMC pulsed current frequency from the MDMC electrotherapy pulsed frequency generator 110 via an electrically conductive cable or wire 110.

The practitioner 122 in an embodiment may use the mobile electrode 123 on a physical manipulation tool like a electrotherapy glove to perform a physical manipulation of the patient's soft tissue or musculature within the physical manipulation target region 131 during delivery of the first MDMC pulsed current frequency through to a stationary electrode such as 113 and delivery of the second MDMC pulsed current frequency to the mobile electrode 121 of the electrotherapy glove 120 and paired with a stationary electrode 115. Because each of the first and second MDMC pulsed frequencies may have a magnitude of current above one mA, the combined application of these first and second MDMC pulsed frequencies may produce visible or noticeable effects during the electrotherapy session. For example, application of these MDMC pulsed frequencies may produce contraction or relaxation of the muscles within the patient's physical manipulation target region 131. This may occur in embodiments where the tissue targeted for treatment (and associated with the practitioner-selected DMC pulse frequencies described in Table 2 below) comprise muscle tissues. This may also occur in embodiments where the tissue targeted for treatment (and associated with the practitioner-selected DMC pulse frequencies described in Table 2 below) comprise non-muscle tissues (e.g., bone, bursa, cartilage, tendon, etc.) situated nearby the muscles producing such observable contraction or relaxation upon application of the first and second MDMC pulsed frequencies.

In some cases, application of these MDMC pulsed frequencies may cause rhythmic contractions of the muscles within the physical manipulation target region 131 at a rate that does not match or correlate to either of the MDMC pulsed frequencies. For example, application of first and second MDMC pulsed frequencies having pulse frequencies (e.g., DC electrical pulses applied at the practitioner-selected frequencies described in Table 2 below, mixed with an AC applied current frequency) above 10 Hz may produce an observable rhythmic contraction of the patient's muscle within the physical manipulation target region 131 at a frequency below 5 Hz. Because the frequency of these contractions may fall well below the pulse frequencies of the applied MDMC pulsed frequencies, one may conclude that the rhythmic contraction of the patient's muscle is not directly caused by the application of these MDMC pulsed frequencies to the physical manipulation target region 131 via the paired stationary and mobile electrodes via electrotherapy glove 120. Rather, the rhythmic contraction of the patient's muscle within the physical manipulation target region 131 indicates a response by the patient's tissues (e.g., muscle or non-muscle tissues targeted for treatment using the practitioner-selected frequencies described in Table 2 below) to the application of these MDMC pulsed frequencies.

A patient's tissues tend to produce these rhythmic contractions within the physical manipulation target region 131 upon application of MDMC pulsed frequencies having MDMC pulse frequencies specifically targeted toward healing of the specific tissues nearby the muscles undergoing these contractions, or targeting a specific condition affecting the tissues nearby the muscles undergoing these contractions. In other words, use of MDMC pulsed frequencies provides real-time and observable feedback practitioners may use to isolate frequencies of applied MDMC pulsed frequencies to which the patient's tissues within the physical manipulation target region 131 respond positively. In such a way, the practitioner in an embodiment may use the MDMC electrotherapy system 100 to determine the optimal frequency for treating a patient's condition and the optimal frequency for treating a patient's tissues within the physical manipulation treatment region 131 affected thereby during a single session, reducing cost and healing time. Further, the electrotherapy with a first or second MDMC pulsed frequency may assist the physical manipulation therapy with location as well as the benefits of electrotherapy with physical manipulation for treatment of a tissue type as well as an identified condition.

As described above, such an electrotherapy-assisted physical manipulation session in an embodiment may begin with a practitioner identifying the physical manipulation target region 131, based on patient's 130 symptoms, complaints, or previous diagnoses. Following identification of the physical manipulation target region 131 in an embodiment, the practitioner may identify a diagnostic amplitude for the first MDMC pulsed current frequency and the second MDMC pulsed current frequency that may be used to isolate optimal MDMC pulse frequencies for treatment of the patient's condition and tissue types affected thereby. For example, the practitioner 122 may apply one or more stationary electrodes 113 or 115 within the physical manipulation target region 131, and initially instruct the MDMC electrotherapy pulsed frequency generator 110 to produce pre-diagnostic first and second MDMC pulsed frequencies having amplitudes (e.g., at or below one Volt) relatively lower than that routinely used during therapeutic physical manipulation for application through mobile electrodes 123 and 121 paired to the stationary electrodes 113 and 115. The practitioner 122 in such an embodiment may then apply the pre-diagnostic first MDMC pulsed current frequency to the patient's physical manipulation target region 131 via the electrode pairs 113 and 123, and apply the pre-diagnostic second MDMC pulsed current frequency via contact between the electrode pairs 115 and 121 also within the physical manipulation target region 131. The practitioner 122 in an embodiment may then slowly increase the amplitudes (e.g., voltage) of the applied first and second MDMC pulsed frequencies until the patient reports, or the practitioner observes a contraction of the patient's tissues within the physical manipulation target region 131. This observation may indicate a diagnostic amplitude (e.g., voltage) the practitioner may use to instruct the MDMC electrotherapy pulsed frequency generator 110 to generate first and second MDMC pulsed frequencies for use in diagnosis or treatment of a condition affecting tissues within the patient's 130 physical manipulation target region 131.

In some embodiments, the patient 130 may be suffering from an identified condition affecting known or previously identified tissues within the physical manipulation target region 131. For example, a patient 130 may have undergone imaging or other diagnostic techniques of tissues within the physical manipulation target region 131 to determine a condition such as a tear, fracture, arthritic inflammation, or chronic overuse, for example is affecting a specifically identified tissue such as a muscle, tendon, ligament, or cartilage. In such an example embodiment, the practitioner may use the MDMC electrotherapy pulsed frequency generator 110 to adjust frequencies of the first and second MDMC pulsed frequencies (e.g., as described in greater detail with respect to FIG. 2, below) to address the identified or known condition affecting the specifically identified tissue type.

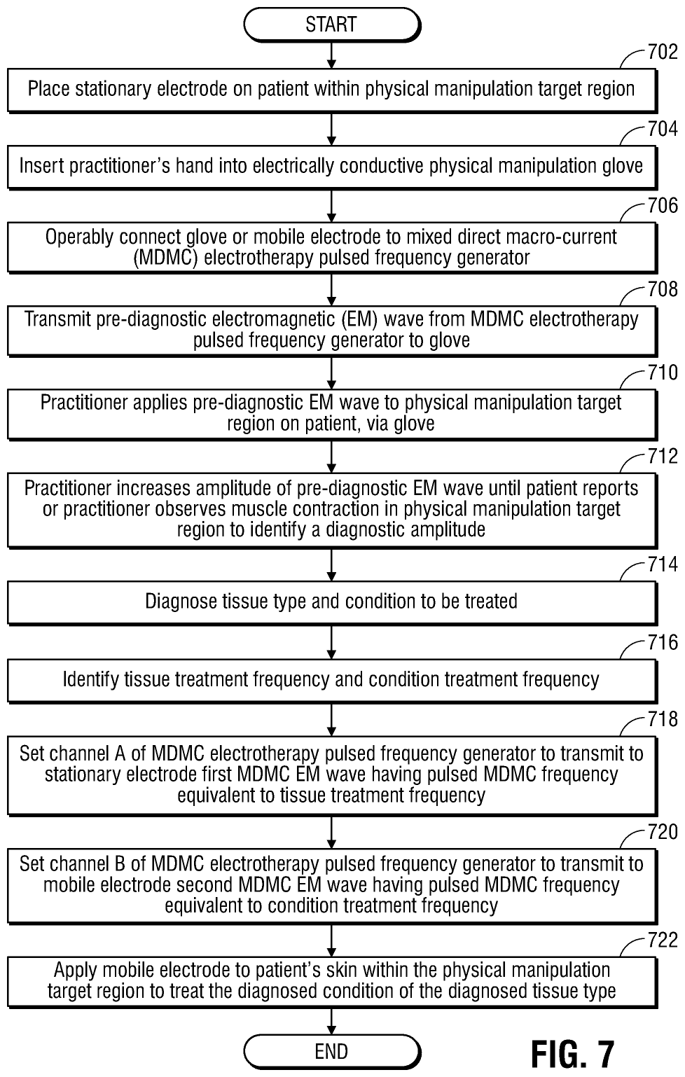

As described in greater detail below with respect to FIG. 7, the first and second MDMC pulsed frequencies may be generated by the MDMC electrotherapy pulsed frequency generator 110 by mixing a direct current (DC) electrical pulse with a magnitude of current over one mA (e.g., direct macrocurrent (DMC)) with an AC applied current frequency designed to inhibit ionization of the patient's tissues that may cause a painful or prickling sensation. The frequency of the DMC electrical pulses in an embodiment may be targeted to treatment of either the identified condition, or the specifically identified tissue affected thereby. By mixing an AC applied current frequency with these DMC electrical pulses having practitioner-specified DMC pulse frequencies in an embodiment, the MDMC electrotherapy pulsed frequency generator 110 may produce an MDMC pulsed current frequency. The frequency of the DMC electrical pulses underlying the first MDMC pulsed current frequency may be targeted toward treatment of the identified condition in an embodiment. The frequency of the DMC electrical pulses underlying the second MDMC pulsed current frequency in such an embodiment may be targeted toward treatment of the tissue type known to be affected by this identified condition. Although FIG. 1 describes delivery of the first MDMC pulsed current frequency to the stationary electrode 113 and mobile electrode 123 and delivery of the second MDMC pulsed current frequency to the stationary electrode 115 and mobile electrode 121, it is contemplated that the reverse scenario may also be employed or any combination of paired electrodes whether stationary or mobile may be used. In other words, if the applied current frequency is a second MDMC pulsed frequency applied to the patient 130 via the stationary electrode 113 and mobile electrode 123 and is targeted toward treatment of the patient's condition, it is contemplated that the applied current frequency applied via the stationary electrode 115 and mobile electrode 121 is a first MDMC pulsed frequency targeted toward the tissue type affected by that condition.

As described herein, many identified conditions and tissue types respond to electrotherapy treatment at specific DMC pulse frequencies underlying the MDMC pulsed frequencies applied to the patient within the physical manipulation treatment region 131. Mixing these DMC pulses with the AC applied current frequency in an embodiment may generate an MDMC pulsed current frequency, such as one of the frequencies described in Table 1 below, to treat identified conditions. MDMC pulse frequencies are associated with specific conditions in such a way that they are referred to herein as condition treatment pulse frequencies. As shown in Table 1, some conditions may be associated with a plurality of MDMC pulse frequencies. Some patients afflicted with these conditions may respond positively to only one of these various frequencies. In some embodiments, even when a patient's condition is known or previously identified, the practitioner may need to identify which of these various frequencies associated with that previously identified condition will most optimally treat a specific patient, as described in greater detail with respect to FIG. 5, below.

TABLE 1

| | | | Condition |
|---|---|---|---|
| | Condition | | Treatment |
| Physical | Treatment | Physical | Pulse |
| Condition | Pulse Frequency | Condition | Frequency |
| General | 294 Hz; 321 | Fibrosis | 51 Hz |
| Maintenance | Hz; 9 Hz | | |
| Chronic | 284 Hz | Bony tissue | 58 Hz; 1 Hz |
| Inflammation or | | scarring | |
| Blood Clot | | | |
| Concussion | 94 Hz | Soft tissue | 58 Hz; 2 Hz |
| | | scarring | |
| Cystic | 59 Hz | Adhesive | 58 Hz; 32 |
| Condition | | scarring | Hz; 13 Hz |
| Emotional | 970 Hz | Muscular | 98 Hz; 46 |
| Component | | paralysis | Hz; 77 Hz |
| Fibrosis | 51 Hz | Nerve | 20 Hz |
| | | pressure/pain | |
| Inflammation, | 40 Hz; 30 | Nerve shock | 94 Hz |
| irritation, pain | Hz; 20 Hz | | |
| reaction | | | |
| Paralysis | 321 Hz | Pain, pressure | 20 Hz; 91 Hz |
| Sclerosis, scar | 3 Hz; 10 Hz; | Agitans paralysis | 59 Hz; 98 Hz |
| | 13 Hz; | | |
| Spasm | 29 Hz | Bleeding/ | 18 Hz |
| | | Hemorrhage | |
| Tear or break | 124 Hz | Spinal cord | 20 Hz; 10 |
| | | pressure | Hz; 11 Hz |
| Trauma | 294 Hz | Spinal pressure, | 20 Hz; 11 Hz |
| | | vertebral | |
| Ulceration | 150 Hz; 750 Hz | Tension | 41 Hz |
| Uric Acid | 8 Hz | Tissue | 58 Hz |
| Buildup | | degeneration | |
| Arthritic toxin | 45 Hz | Tissue | 681 Hz |
| | | encapsulation | |
| Tissue fibrosis | 51 Hz; 105 Hz; | Tissue necrosis | 54 Hz |
| | 601 Hz | | |
| Lipoma | 77 Hz | Ulceration | 150 Hz |
| Vagus nerve | 109 Hz; 90 Hz; | Increase | 81 Hz |
| irregularity | 33 Hz | Secretion | |

Table 2 below provides an example of a plurality of specific MDMC pulse frequencies that treat specific tissue types. MDMC pulse frequencies known to be associated with specific conditions in such a way may be referred to herein as tissue treatment pulse frequencies. As shown in Table 2, some tissue types may also be associated with a plurality of pulse frequencies. Some patients with conditions affecting these tissue types may respond positively to only one of these various frequencies. In some embodiments, even when the tissue type affected by a condition has been previously identified, the practitioner may need to identify which of these various frequencies associated with that previously identified tissue type will most optimally treat a specific patient, as described in greater detail with respect to FIG. 5, below.

TABLE 2

| | Tissue Treatment | | Tissue Treatment |
|---|---|---|---|
| Tissue Type | Pulse Frequency | Tissue Type | Pulse Frequency |
| Spinal Cord | 10 Hz | Adipose tissue | 97 Hz |
| Vertebra | 11 Hz | Bone | 59 Hz; 39 Hz |
| Fibroid tumor | 51 Hz; 105 Hz; | Bursa | 195 Hz |
| | 601 Hz | | |
| Lipoma | 77 Hz | Cartilage | 157 Hz |
| Vagus nerve | 109 Hz | Connective Tissue | 77 Hz |
| Disc | 330 Hz; 630 Hz; | Fascia, muscle | 142 Hz |
| | 710 Hz | sheath | |
| Joint capsule | 480 Hz | Ligament | 100 Hz |
| Lymphatic tissue | 13 Hz | Muscle Tissue | 46 Hz |
| Peripheral Nerve | 396 Hz | Periosteum | 783 Hz |
| tissue | | | |
| Skin tissue | 355 Hz | Bony Spine | 11 Hz |
| Tendon | 191 Hz | Vein | 79 Hz |
| Sacral nerve | 78 Hz | Solar nerve | 200 Hz |
| plexus | | plexus | |
| Artery | 62 Hz | Immune System | 116 Hz |

Several of the above tissue and condition frequencies are established though work done with microcurrent electrotherapy. As described herein, upon application of a first MDMC pulsed current frequency having an MDMC pulse frequency specifically targeting a specific condition, and application of a second MDMC pulsed current frequency affecting the tissues affected by that condition, those tissues or the muscles nearby those tissues may undergo rhythmic contractions at a frequency far below either of the MDMC pulse frequencies. These rhythmic contractions may be observable by the practitioner as an indication that the proper combination of an optimal MDMC pulse frequency for treatment of the condition and an optimal MDMC pulse frequency for treatment of the tissue type affected thereby have been isolated. However, these rhythmic contractions may only occur in an embodiment when the first and second MDMC pulsed frequencies applied to the patient have the optimal MDMC pulse frequency for that patient's condition and the optimal MDMC pulse frequency for the tissue type affected thereby. In the case where either or both of the identified or known condition or tissues affected thereby are associated with a plurality of frequencies (e.g., as shown in Tables 1 and 2 above), the practitioner may apply a plurality of combinations of these frequencies in order to identify the optimal MDMC pulse frequencies for treatment of the condition and the tissue type for that specific patient, as described in greater detail with respect to FIG. 5, below. As described herein, the practitioner may determine the optimal MDMC pulse frequency for treatment of the patient's condition and the tissues affected by that condition have been isolated when the tissues or the muscles nearby the patient's tissues within the physical manipulation treatment region 131 undergo a rhythmic contraction at a frequency (e.g., below 5 Hz) that is well below either of the applied MDMC pulse frequencies (e.g., above 10 Hz).

The frequencies associated with the physical conditions listed within Table 1 and the frequencies associated with the tissue types listed within Table 2 may be estimates or may represent median values within a range of values known to treat associated conditions or tissue types. The conditions listed in Table 1 may be treated in various embodiments described herein through application of an MDMC pulsed current frequency having an MDMC pulse frequency that is within 3-5 Hz of the frequency associated with each condition within Table 1. For example, the physical condition of tension may be treated using a range of MDMC pulse frequencies between 36 Hz and 46 Hz, having a median frequency of 41 Hz listed in Table 1. Similarly, the tissue types listed in Table 2 may be treated in various embodiments described herein through application of an MDMC pulsed current frequency having an MDMC pulse frequency that is within 5 Hz of the frequency associated with each tissue type within Table 2. For example, the lipoma tissue type may be treated using a range of MDMC pulse frequencies between 72 Hz and 82 Hz, having a median frequency of 77 Hz listed in Table 2.

For example, in an embodiment in which the patient 130 has been identified with fibrosis of a joint capsule, the practitioner may refer to Tables 1 and 2 above to determine the optimal MDMC pulse frequency of 51 Hz may treat the identified condition of fibrosis and the optimal MDMC pulse frequency of 480 Hz may treat the joint capsule tissue type affected by the fibrotic condition. In such an embodiment, the practitioner may observe the rhythmic contractions described directly above upon application of a first MDMC pulsed current frequency having an MDMC pulse frequency of 51 Hz and application of a second MDMC pulsed current frequency having an MDMC pulse frequency 480 Hz.

As another example, in an embodiment in which the patient 130 has been identified with inflammation affecting a bursa of one of the patient's joints, the practitioner may refer to Tables 1 and 2 above to determine the optimal MDMC pulse frequency of 40 Hz, 30 Hz, or 20 Hz may treat the identified condition of inflammation and the optimal MDMC pulse frequency of 195 Hz may treat the bursa tissue type affected by such inflammation. In such an embodiment, the practitioner may observe the rhythmic contractions of those tissues or the muscles nearby described directly above upon application of a first MDMC pulsed current frequency having an MDMC pulse frequency of 40 Hz and application of a second MDMC pulsed current frequency having an MDMC pulse frequency 195 Hz. The practitioner may not observe the rhythmic contractions of those tissues or the muscles nearby in such an embodiment when the first MDMC pulsed current frequency is set to an MDMC pulse frequency of 30 Hz or 20 Hz, indicating the specific patient 130 may respond more positively to the MDMC pulse frequency of 40 Hz, rather than 30 Hz or 20 Hz for treatment of the identified inflammation.

In still another example, in an embodiment in which the patient 130 has been identified with a fracture affecting bone tissue, the practitioner may refer to Tables 1 and 2 above to determine the optimal MDMC pulse frequency of 124 Hz may treat the identified condition of a fracture or break and the optimal MDMC pulse frequency of 59 Hz or 39 Hz may treat the bone tissue affected by such fracture. In such an embodiment, the practitioner may observe the rhythmic contractions of muscles situated nearby the fractured bone described directly above upon application of a first MDMC pulsed current frequency having an MDMC pulse frequency of 124 Hz and application of a second MDMC pulsed current frequency having an MDMC pulse frequency 59 Hz. The practitioner may not observe the rhythmic contractions in the muscles situated nearby the fractured bone in such an embodiment when the second MDMC pulsed current frequency is set to an MDMC pulse frequency of 39 Hz, indicating the specific patient 130 may respond more positively to the MDMC pulse frequency of 59 Hz, rather than 39 Hz for treatment of the bone tissue affected by such fracture.

As yet another example, in an embodiment in which the patient 130 has been identified with vertebral spinal pressure affecting a patient's vertebral disc(s), the practitioner may refer to Tables 1 and 2 above to determine the optimal MDMC pulse frequency of 20 Hz, or 11 Hz may treat the identified condition of vertebral spinal pressure and the optimal MDMC pulse frequency of 330 Hz, 630 Hz, or 710 Hz may treat the vertebral disc tissue type affected by the vertebral spinal pressure condition. In such an embodiment, the practitioner may observe the rhythmic contractions of muscles nearby the vertebral disc described directly above upon application of a first MDMC pulsed current frequency having an MDMC pulse frequency of 20 Hz and application of a second MDMC pulsed current frequency having an MDMC pulse frequency 330 Hz. The practitioner may not observe the rhythmic contractions in muscles nearby the vertebral disc in such an embodiment when the first MDMC pulsed current frequency is set to an MDMC pulse frequency of 630 Hz or 710 Hz, indicating the specific patient 130 may respond more positively to the MDMC pulse frequency of 330 Hz, rather than 630 Hz or 710 Hz for treatment of the identified vertebral spinal pressure condition. Similarly, the practitioner may not observe the rhythmic contractions in such an embodiment when the second MDMC pulsed current frequency is set to an MDMC pulse frequency of 11 Hz, indicating the specific patient 130 may respond more positively to the MDMC pulse frequency of 20 Hz, rather than 11 Hz for treatment of the vertebral disc tissue affected by the vertebral spinal pressure condition.

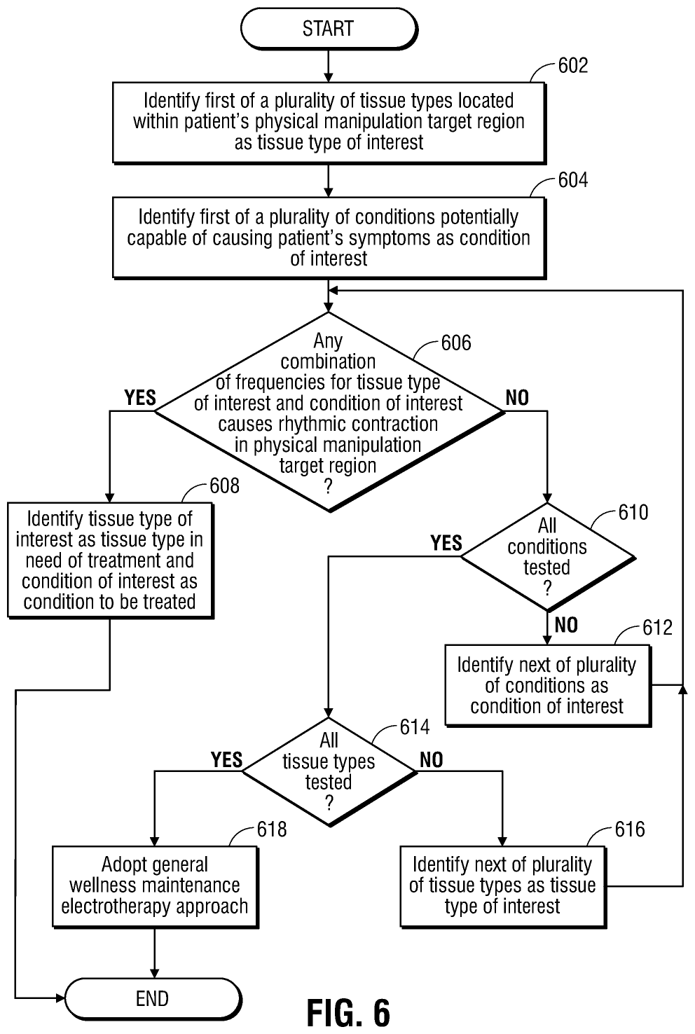

In some embodiments, the practitioner may use the MDMC electrotherapy system 100 to diagnose an unknown condition affecting potentially several tissues not yet specifically identified within the patient's physical manipulation target region 131. As described herein, the combined application of a first MDMC pulsed current frequency targeted toward treatment of an existing but undiagnosed condition and application of a second MDMC pulsed current frequency targeted toward treatment of the patient's tissues affected by that condition within the patient's physical manipulation target region 131 may produce rhythmic contraction of the patient's 130 muscle within the physical manipulation target region 131. This rhythmic contraction may occur at a frequency (e.g., below 5 Hz) below the MDMC pulse frequencies (e.g., above 10 Hz) of either the first or second MDMC pulsed frequencies so applied to the physical manipulation target region 131. Observance of these rhythmic contractions in an embodiment may indicate that the practitioner has isolated the MDMC pulse frequency optimal for treatment of the patient's condition and the MDMC pulse frequency optimal for treatment of the patient's tissues affected by that condition. Once these frequencies have been isolated, as described in greater detail with respect to FIG. 6, below, the practitioner may then cross-reference the known conditions and tissues responsive to these frequencies (e.g., through reference to Tables 1 and 2 above) to diagnose the patient's condition and specifically identify the tissue types affected by that condition. As also described herein, the tissue type affected by that condition determined in such a way may be muscle tissue or non-muscle tissue such as bursa, cartilage, bone, tendon, etc.

For example, in an embodiment in which the practitioner has observed these rhythmic contractions upon application of a first MDMC pulsed current frequency having a first MDMC pulse frequency and application of a second MDMC pulsed current frequency having a second MDMC pulse frequency, the practitioner may refer to Tables 1 and 2 above to identify the patient's condition and tissues affected thereby based on these first and second MDMC pulse frequencies. In such an example embodiment, if the first MDMC pulse frequency is associated with only one possible condition and the second MDMC pulse frequency is associated with only one possible tissue type potentially affected thereby, the practitioner may determine the condition and tissue type affecting that tissue type. For example, in an embodiment, the practitioner may observe rhythmic contractions of the patient's muscle within the physical manipulation target region upon application of a first MDMC pulsed current frequency having an MDMC pulse frequency of 284 Hz, which is associated only with the condition of chronic inflammation or blood clot (e.g., as shown in Table 1) and application of a second MDMC pulsed current frequency having an MDMC pulse frequency of 157 Hz, which is associated only with the cartilage tissue type (e.g., as shown in Table 2). In such an embodiment, the practitioner may determine, based on the observed rhythmic contractions of the muscles within the physical manipulation target region and known MDMC pulse frequencies of the first and second MDMC pulsed frequencies that the patient is suffering from chronic inflammation or blood clot within a cartilage situated nearby the muscle undergoing contraction. In an embodiment in which the patient has not been diagnosed and only complains of generalized pain surrounding a major joint (e.g., knee or shoulder), this method may be used to rule out other conditions and tissues that may cause such generalized pain, such as scarring, nerve pain, cysts, tear, break, degeneration, necrosis, or ulceration of tissues within that joint other than the cartilage, such as bone, adipose or soft tissue, bursa, tendon, ligament, connective tissue, muscle tissue, nerve tissue, lymphatic tissue, or veins.

Upon identification of the optimal MDMC pulse frequency for the patient's identified condition and identification of the optimal MDMC pulse frequency for the patient's tissue type affected by that identified condition in an embodiment, the practitioner may initiate an electrotherapy-assisted physical manipulation treatment session. In such a session, the practitioner may set the MDMC electrotherapy pulsed frequency generator 110 to transmit a first MDMC pulsed current frequency having a current magnitude over one mA and an MDMC pulse frequency matching the optimal frequency for the patient's identified condition (e.g., as listed in Table 1 as a condition treatment pulse frequency) to an electrode pair. The practitioner in such an embodiment may also set the MDMC electrotherapy pulsed frequency generator 110 to transmit a first MDMC pulsed current frequency having a current magnitude over one mA and an MDMC pulse frequency matching the optimal frequency for the patient's tissue type (e.g., as listed in Table 2 as a tissue treatment pulse frequency) affected by the identified condition to a second electrode pair. Each electrode pair may include a mobile electrode of a physical manipulation tool such as an electrotherapy glove.

The practitioner may then apply the mobile electrode or electrodes via an electrotherapy glove 120 to the patient's skin within the physical manipulation target region 130. This may occur along with physical manipulation of tissues in the target region 131 by a practitioner. The combined application of the first MDMC pulsed current frequency targeted toward treatment of the identified condition and application of the second MDMC pulsed current frequency targeted toward treatment of the tissues affected by the identified condition within the patient's physical manipulation target region 131 in such an embodiment may produce rhythmic contraction of the patient's 130 muscles within the physical manipulation target region 131 which the practitioner can sense during a physical manipulation of a patient via electrotherapy glove 120. In other words, use of MDMC pulsed frequencies provides real-time and observable feedback practitioners may use to isolate frequencies of applied MDMC pulsed frequencies to which the patient's tissues respond positively. This may also provide greater location accuracy for treatment and physical manipulation within the broader physical manipulation target region 131. The MDMC electrotherapy system 100 in an embodiment thus allows a practitioner to determine the optimal frequency for treating a patient's condition and the optimal frequency for treating a patient's tissues affected thereby during a single session, reducing cost and healing time. The MDMC electrotherapy system 100 may also assist physical manipulation therapy in connection with electrotherapy for specific location and mutual application of electrotherapy pulsed current frequencies with chiropractic physical manipulation via the practitioner using an electrotherapy glove or other physical manipulation tools.

FIG. 2 is a graphical diagram illustrating internal components of a mixed direct macro-current (MDMC) electrotherapy pulsed frequency generator 201 for transmitting an MDMC pulsed current frequency to a stationary electrode and a mobile electrode during an electrotherapy-assisted physical manipulation treatment according to an embodiment of the present disclosure. The MDMC electrotherapy pulsed frequency generator 201 may be powered in an embodiment using standard 120V 60 Hz AC power via an external wall-mount power supply or internal AC-to-DC conversion. The mixed direct macro-current (MDMC) electrotherapy pulsed frequency generator 201 in an embodiment may have a master shut-off switch 202 that may be modular in that each channel may be controlled individually through panel-mount controls. Channel outputs 204A and 204B in an embodiment may each provide the sum of two waveforms, an AC applied current frequency mixed with a direct macrocurrent (DMC) electrical pulse applied at either a condition treatment pulse frequency (shown in Table 1 above) or at a tissue treatment pulse frequency (shown in Table 2 above), as described in greater detail below with respect to FIG. 5. Mixing the DMC electrical pulses with an AC applied current frequency in such a way in an embodiment may generate a mixed DMC (MDMC) applied current frequency having an MDMC pulse frequency equivalent to either the condition treatment pulse frequency or the tissue treatment pulse frequency, as described herein.

In a first embodiment, the MDMC pulsed current frequency of channel port 204A may have an MDMC pulse frequency targeted toward treatment of a patient's condition and the MDMC pulsed current frequency of channel port 204B may have an MDMC pulse frequency targeted toward treatment of a patient's tissue type affected the patient's condition. In a second embodiment, the MDMC pulsed current frequency of channel port 204B may have an MDMC pulse frequency targeted toward treatment of a patient's condition and the MDMC pulsed current frequency of channel port 204A may have an MDMC pulse frequency targeted toward treatment of a patient's tissue type affected the patient's condition.

The channel ports 204A and 204B may be interchangeably connected to either pair of electrodes whether those electrode pairs are stationary electrodes, mobile electrodes, or some combination of electrode types (e.g., as described with respect to FIG. 1, above). For example, in a first embodiment, the MDMC electrotherapy pulsed frequency generator 201 may be operably connected via channel port 204A to an electrode pair including a stationary electrode 113 and mobile electrode 123 operably connected via an electrically conductive wire 112 as shown in FIG. 1. Also, the MDMC electrotherapy pulsed frequency generator 201 may be operably connected via channel port 204B to a stationary electrode 115 and a mobile electrode 121 and operably connected via an electrically conductive wire 111 as shown in FIG. 1. As another example, in a second embodiment, the MDMC electrotherapy pulsed frequency generator 201 may be operably connected to those pairs of electrodes via switched channel ports 204A and 204B, in other words, opposite coupling of wires 111 and 112 to ports 204A and 204B for the electrode pairs shown in FIG. 1. The channel ports 204A and 204B may also receive a return applied current frequency from one of the electrode pairs.

A knob 203 or other input device such as a slider 205 in an embodiment may be used in order to adjust an amplitude or a frequency of an MDMC pulsed current frequency in an embodiment. For example, a knob 203 or slider 205, either of which may be mechanical or displayed within a touchscreen, or other input control may be used by a practitioner in an embodiment to set a pulsed DMC electrical condition treatment applied current frequency to match an MDMC pulse frequency for treatment of a patient's physical condition. As another example, knob 203 or slider 205 may be used by the practitioner in such an embodiment to set a pulsed DMC electrical tissue treatment applied current frequency to match an MDMC pulse frequency for treatment of the patient's tissue affected by that physical condition. A digital processor in an embodiment may synthesize the waveforms for output and pass the results through digital-to-analog conversion and amplification stages. The waveform or visual representation of the resulting MDMC pulsed current frequency output via port 204A or 204B and adjusted via knob 203 or slider 205 may be displayed via a liquid crystal display (LCD), organic light emitting diode (OLED), or other display screen 206 in an embodiment. In other embodiments, the display screen 206 may include a control graphical user interface (GUI) for input and output relating to commands to control frequency, amplitude or other features of the first or second MDMC pulsed frequencies with AC current components in various embodiments. The display screen 206 may be touch display screen to receive inputs in various embodiments. Additionally the display screen 206 may display the pulsed frequencies, settings, time, or other data relating to mixed electrotherapy and physical manipulation therapy. Controls on the applied MDMC pulsed frequencies may include an output amplitude cap in an embodiment that may be performed on the processor, for example, and also be clamped by hardware for redundancy. Firmware developed using development environments MPLAB X and Microchip in parallel with the sourcing of the assembled circuit boards may be used in an embodiment.

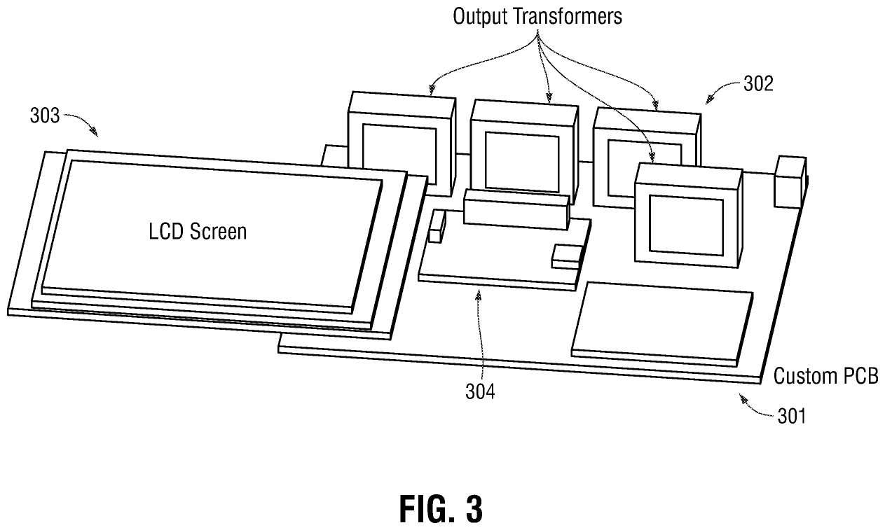

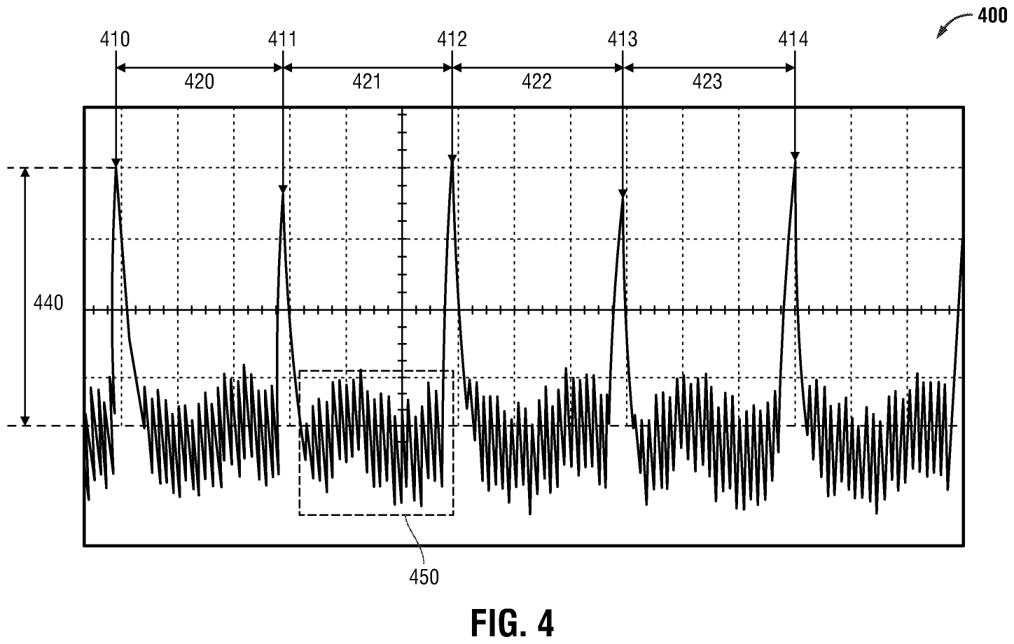

FIG. 3 is a graphical diagram illustrating internal components of a mixed direct macro-current (MDMC) electrotherapy pulsed frequency generator for transmitting a repeating DC electrical pulse to an electrotherapy glove during physical manipulation of a patient's musculature by the electrotherapy glove according to an embodiment of the present disclosure. A printed circuit board (PCB) 301 in an embodiment may utilize digital applied current frequency synthesis for creation of the MDMC pulsed current frequency by routing electrical power through a combination of available infrastructure described in greater detail below with respect to FIG. 4. Controller 304 in an embodiment may receive input from a practitioner (e.g., as described above with respect to FIG. 2 through movement of various knobs, sliders, GUI touchscreen controls, or others for adjusting MDMC pulse frequencies of a first MDMC pulsed current frequency transmitted via a first channel port (e.g., channel port 204A of FIG. 2) and of a second MDMC pulsed current frequency transmitted via a second channel port (e.g., channel port 204B of FIG. 2).

The power stage in an embodiment may consist of an amplifier, such as a signal or audio amplifier, driving output transformers 302 in FIG. 3 which may isolate the user electrodes from the digital electronics, earth ground, and input power. This stimulation topology in an embodiment may offer a wide range of possibilities regarding applied, pulsed or AC current frequency, amplitude, and mixture, input power requirements, and output power levels, lending to efficient design of instruments meeting varying application requirements. For example, applied, pulsed or AC current frequency, amplitude, and mixture can be changed in firmware by simply reprogramming the device. Output power can be adjusted by either scaling the amplitude of the synthesized applied, pulsed or AC current frequency, or by reducing the amplification of the audio amplifier. LCD screen 303 and corresponding LCD internal components can be programmed to offer various pictures of electrode placements on the patient or videos of various physical manipulations or exercise movements that correspond with various exercises and routines consistent with descriptions in this disclosure.

FIG. 4 is a graphical diagram illustrating an MDMC pulsed current frequency 400 formed by mixing a pulsed DMC component and an AC applied current frequency component according to an embodiment of the present disclosure. Various characteristics of the MDMC pulsed current frequency 400 displayed at FIG. 4 may be tailored toward treatment of a patient's identified condition or treatment of a patient's tissue types affected by that condition in various embodiments, as described in greater detail with respect to FIG. 7, below. In further embodiments, various characteristics of the MDMC pulsed current frequency 400 may be adjusted by a practitioner as part of a method of diagnosing an unknown condition affecting one of a plurality of possible tissue types, as described in greater detail below with respect to FIG. 6. As described herein, plural MDMC pulsed frequencies 400, also referred to as MDMC pulsed current frequencies, may be applied via plural sets of electrodes to target tissues as well as conditions as described in various embodiments herein. These MDMC pulse frequencies 400 may have varied components including pulsed frequency, amplitude and AC frequency components in variations of embodiments herein.

The MDMC pulsed current frequency shown in FIG. 4 may represent an MDMC pulsed current frequency 400 targeted toward treatment of a specific condition in a first embodiment. This MDMC pulsed current frequency 400 may include an underlying direct macrocurrent (DMC) electrical pulse component applied at an MDMC pulse frequency known to treat a specific physical condition (e.g., a condition treatment pulse frequency, as shown in Table 1, above). For example, FIG. 4 may depict an MDMC pulsed current frequency 400 targeted toward treatment of a concussion (as shown in Table 1 above) in an embodiment in which peaks 410, 411, 412, 413, and 414 occur at a rate or frequency of 94 peaks per second or 94 Hz.

In a second embodiment, the MDMC pulsed current frequency 400 shown in FIG. 4 may represent an MDMC pulsed current frequency 400 targeted toward treatment of a specific tissue type affected by such a physical condition. This MDMC pulsed current frequency 400 may include an underlying a DMC electrical pulse applied at an MDMC pulse frequency 400 targeted to treat a specific tissue type (e.g., a tissue treatment pulse frequency, as shown in Table 2, above). For example, FIG. 4 may depict an MDMC pulsed current frequency 400 targeted toward treatment of a bursa (as shown in Table 2 above) in an embodiment in which peaks 410, 411, 412, 413, and 414 occur at a rate or frequency of 195 peaks per second or 195 Hz.

As described herein, existing electrotherapy systems apply an electrical current to a patient's musculature or soft tissue via one or more electrodes or similar structures placed in contact with a patient's skin. Some systems apply a repeating DC electrical pulse because DC electrical pulses at high frequencies (e.g., 500 to 1000 Hz) have been shown to decrease muscle contraction. However, such DC electrical pulses may cause a Faradaic reaction within the patient's skin or adipose tissue situated nearby or in contact with the electrode delivering the DC electrical pulse, in which charged particles (e.g., electrons or ions) transfer across the electrode and into the patient's skin or tissue. These ions may then reduce or oxidize to another species, which may further cause a patient to experience a prickling, hot, or painful sensation at the site of application. Some existing electrotherapy systems instead employ an AC applied current frequency, rather than a DC electrical pulse. Alternating current applied current frequencies inhibit the transfer of charged particles across the electrode and into the patient's skin or adipose tissue. Thus, application of an AC applied current frequency decreases or inhibits the prickling, hot, or painful sensations felt by patients during application of direct current.

The MDMC electrotherapy system in embodiments of the present disclosure may generate an MDMC pulsed current frequency 400 comprised of a repeating DC electrical pulse component mixed with an AC applied current frequency component to attain the benefits of each. The MDMC pulsed current frequency 400 may include DMC pulses 410, 411, 412, 413, and 414 of peak amplitude 440 (e.g., between 4 and 5 Volts) occurring at regular intervals (e.g., 420, 421, 422, 423, respectively) preset or adjustable by the practitioner. As described herein, the practitioner in an embodiment may apply to a patient's skin within a physical manipulation target region a first MDMC pulsed current frequency with a first MDMC pulse frequency targeted toward treatment of the patient's identified condition, and a second MDMC pulsed current frequency with a second MDMC pulse frequency targeted toward treatment of the patient's tissue affected by the identified condition, as discussed in greater detail below with respect to FIGS. 6 and 8, below. For example, the MDMC pulsed current frequency 400 in an embodiment may be pulsed at a frequency (e.g., between one and 1,000 Hz) associated with a identified condition (e.g., as shown in Table 1 above), or associated with a tissue type (e.g., as shown in Table 2 above).

An AC applied current frequency component may be superimposed or mixed with this MDMC pulsed current frequency in embodiments described herein in order to inhibit the transfer of charged particles across electrodes carrying the applied current frequency and applied to the patient. The AC applied current frequency component may be mixed in with the MDMC pulsed current frequency between each of the pulses 410, 411, 412, 413, and 414 in an embodiment, as shown within box 450. The AC applied current frequency component have an amplitude magnitude anywhere between zero and one Volt. In some embodiments, as shown in FIG. 4, the amplitude of the AC applied current frequency component may vary between a positive and negative value, with the median amplitude at zero volts (e.g., between negative 0.5 Volts and positive 0.5 Volts). The AC applied current frequency component may have a frequency relatively higher than the frequency of the MDMC pulsed current frequency 400 in an embodiment. For example, the AC applied current frequency component may have a frequency anywhere between one and 1,000 Hz, as compared to the frequency range of one to 1,000 Hz (or 0.001 and one KHz) for the MDMC pulsed current frequency 400. In a specific embodiment, the AC applied current frequency component may have a frequency of 40 KHz and the MDMC pulsed current frequency 400 may have a DMC component pulsed frequency of 450 Hz.

This combined or mixed applied current frequency that includes both the repeated DMC pulses and an AC applied current frequency may cause relaxation or contraction of the patient's muscles while avoiding the prickling, hot, or painful sensations caused by application of DMC current alone. Mixing of the AC applied current frequency component with the DMC electrical pulse component in such a way may effectively modulate the amplitude (e.g., describing total voltage from both the underlying MDMC pulsed current frequency and the AC applied current frequency) of the MDMC pulsed current frequency 400, resulting in the MDMC pulsed current frequency 400 applied via one or more pairs of electrodes to a patient.

FIG. 5 is a flow diagram illustrating a method of determining a tissue treatment pulse frequency and a condition treatment pulse frequency for applied current frequencies to apply to a patient during treatment of an identified condition and tissue affected by that condition according to an embodiment of the present disclosure. As described herein, and as shown in Tables 1 and 2, some physical conditions of the patient or tissues affected thereby may be associated with a plurality of pulse frequencies. Some patients afflicted with these conditions or tissues affected thereby may respond positively to only one of several available combinations of a frequency for treating the patient's condition and a frequency for treating the tissues affected by that condition. In some embodiments, even when a patient's condition or affected tissue is known or previously identified, the practitioner may need to identify which of these various frequency combinations associated with that previously identified condition or tissue will most optimally treat a specific patient. FIG. 5 provides a method of identifying an optimal combination of such frequencies from a plurality of frequencies known to treat a previously identified condition affecting a previously identified tissue type affected by that condition.

At block 502, the practitioner in an embodiment may identify the first of a plurality of MDMC pulse frequencies associated with a previously identified tissue type affected by a previously identified condition as a tissue treatment pulse frequency of interest. For example, as described above with respect to FIG. 4 depicting an MDMC pulsed current frequency, a frequency or rate with which peaks 410, 411, 412, 413, and 414 occur may represent an MDMC pulse frequency. The MDMC pulsed current frequency in an embodiment may include an MDMC pulse frequency targeted toward treatment of a tissue type known to be affected by a previously identified condition, as described above with respect to Table 2 (e.g., bursa). In such an embodiment, the MDMC pulse frequency listed in Table 2 as being associated with the identified tissue type (e.g., 195 Hz associated with bursa) may show peaks (e.g., 410, 411, 412, 413, and 414) occurring at a rate or frequency of 195 peaks per second or 195 Hz.

As described in an example embodiment with reference to FIG. 1, some tissue types (e.g., as shown in Table 2) may be associated with more than one MDMC pulse frequency targeted toward treatment of that specific tissue. More specifically, with reference to Table 2, tissue types such as vertebral discs may be associated with a plurality of frequencies targeted toward treatment of those tissues (e.g., 330 Hz, 630 Hz, or 710 Hz). In an example embodiment in which the patient 130 has been identified with vertebral spinal pressure affecting a patient's vertebral disc(s), the practitioner may refer to Tables 1 and 2 above to determine the optimal MDMC pulse frequency of 20 Hz, or 11 Hz may treat the identified condition of vertebral spinal pressure and the optimal MDMC pulse frequency of 330 Hz, 630 Hz, or 710 Hz may treat the vertebral disc tissue type affected by the vertebral spinal pressure condition. The practitioner at block 502 in such an embodiment may identify the 330 Hz MDMC pulse frequency as the tissue treatment pulse frequency of interest. The practitioner may then proceed to test this tissue treatment pulse frequency of interest in an embodiment to determine whether it is the optimal MDMC pulse frequency for treatment of the tissue causing the patient's current discomfort or pain.

The practitioner in an embodiment may set channel A of the MDMC electrotherapy pulsed frequency generator to transmit a first applied current frequency to a mobile electrode and stationary electrode pair having an amplitude equivalent to a diagnostic amplitude and the first MDMC pulsed current frequency equivalent to a tissue-type for treatment pulse frequency of interest at block 504. For example, in an embodiment described with reference to FIG. 1, the practitioner in an embodiment may set the MDMC pulse frequency for a first MDMC pulsed current frequency applied to the patient 130 via the stationary electrode 113 and mobile electrode 123 pair to be equivalent to the first of the plurality of tissue treatment pulse frequencies associated with the vertebral disc tissue type in Table 2. More specifically, the practitioner may set the MDMC pulse frequency for a first MDMC pulsed current frequency output on Channel A of the MDMC electrotherapy pulsed frequency generator 110 to a value of 330 Hz.

At block 506, the practitioner in an embodiment may identify the first of a plurality of MDMC pulse frequencies associated with a previously identified condition as a condition treatment pulse frequency of interest. For example, as described above with respect to FIG. 4 depicting an MDMC pulsed current frequency, a frequency or rate with which peaks 410, 411, 412, 413, and 414 occur may represent an MDMC pulse frequency. A second MDMC pulsed current frequency in an embodiment may include an MDMC pulse frequency targeted toward treatment of a previously identified condition, as described above with respect to Table 1 (e.g., concussion). In such an embodiment, the MDMC pulse frequency listed in Table 1 as being associated with the identified condition (e.g., 94 Hz associated with concussion) may show peaks (e.g., 410, 411, 412, 413, and 414) occurring at a rate or frequency of 94 peaks per second or 94 Hz.

As described in an example embodiment with reference to FIG. 1, some identified conditions (e.g., as shown in Table 1) may be associated with more than one MDMC pulse frequency targeted toward treatment of that identified condition. More specifically, with reference to Table 1, physical conditions such as vertebral spinal pressure may be associated with a plurality of frequencies targeted toward treatment of those tissues (e.g., 20 Hz, or 11 Hz). In an example embodiment in which the patient 130 has been identified with vertebral spinal pressure affecting a patient's vertebral disc(s), the practitioner may refer to Tables 1 and 2 above to determine the optimal MDMC pulse frequency of 20 Hz, or 11 Hz may treat the identified condition of vertebral spinal pressure and the optimal MDMC pulse frequency of 330 Hz, 630 Hz, or 710 Hz may treat the vertebral disc tissue type affected by the vertebral spinal pressure condition. The practitioner at block 706 in such an embodiment may identify the 20 Hz MDMC pulse frequency as the condition treatment pulse frequency of interest. The practitioner may then proceed to test this condition treatment pulse frequency of interest in an embodiment to determine whether it is the optimal MDMC pulse frequency for treatment of the physical condition causing the patient's current discomfort or pain.

The practitioner in an embodiment may set channel B of the MDMC electrotherapy pulsed frequency generator to transmit a second applied current frequency to the mobile electrode having an amplitude equivalent to a diagnostic amplitude and a second MDMC pulse frequency equivalent to the condition treatment pulse frequency of interest at block 508. For example, in an embodiment described with reference to FIG. 1, the practitioner in an embodiment may set the MDMC pulse frequency for the second MDMC pulsed current frequency applied to the patient 130 via the mobile electrode 120 to be equivalent to the first of the plurality of condition treatment pulse frequencies associated with the physical condition of vertebral spinal pressure in Table 1. More specifically, the practitioner may set the MDMC pulse frequency for a second MDMC pulsed current frequency output on Channel B of the MDMC electrotherapy pulsed frequency generator 110 to a value of 20 Hz.

At block 510, the practitioner in an embodiment may apply the mobile electrode to the patient's skin within the physical manipulation target region, and may apply the stationary electrode to the patient's skin. For example, the first MDMC pulsed current frequency set at block 504 to output via channel A may be transmitted from the MDMC electrotherapy pulsed frequency generator 110 in an embodiment via an electrically conductive wire 112 to the mobile electrode 123, such as in the electrotherapy glove 120, and the stationary electrode 113 affixed to the patient's 130 skin. In such an example embodiment, the mixed direct macro-current (MDMC) electrotherapy pulsed frequency generator 110 may also transmit a second MDMC pulsed current frequency to a receiving port 121 of a mobile electrode 121 and stationary electrode 115 pair operated by a practitioner via electrical wire 111. The practitioner 122 in an embodiment may use the mobile electrodes 123 and 121, such as in the electrotherapy glove 120, to also perform a physical manipulation of the patient's soft tissue or musculature within the physical manipulation target region 131 during delivery of the first MDMC pulsed current frequency to the stationary electrode 113 and delivery of the second MDMC pulsed current frequency to the port 121 of the mobile electrode 120. Further, during physical manipulation via the electrotherapy glove or the practitioners other hand, response rhythmic muscle contractions of target muscle tissue or muscle tissue near the target treatment tissue may be detected to assist the practitioner with specific location within a physical manipulation target region 131 to provide electrotherapy and physical manipulation or may provide indication that the physical manipulation target region is inaccurate and another region nearby or elsewhere is in need of electrotherapy or physical manipulation treatment.

The practitioner may determine at block 512 in an embodiment whether the patient's muscle within the physical manipulation target region undergoes rhythmic contraction indicating that the optimal treatment pulse frequencies for the previously identified condition affecting the previously identified tissues situated nearby the contracting muscle have been isolated. Moreover, a more specific location may be detected from the responsive rhythmic contractions as well as whether the physical manipulation target region is accurate may be determined at 512. As described herein, upon application of a first MDMC pulsed current frequency having an MDMC pulse frequency specifically targeting a specific tissue-type, or application of a second MDMC pulsed current frequency affecting the condition, those tissues or muscle situated nearby those tissues may undergo rhythmic contractions at a frequency far below either of the MDMC pulse frequencies. These rhythmic contractions may be observable by the practitioner as an indication that the proper combination of an optimal MDMC pulse frequency for treatment of the tissue type and an optimal MDMC pulse frequency for treatment of the condition affecting the tissue have been isolated. The rhythmic contractions may also confirm or redetermine a particular physical manipulation target region and refine a more precise location of a treatment location within a physical manipulation target region as detected by a practitioner via the electrotherapy glove or via a non-gloved hand. However, these rhythmic contractions may only occur in an embodiment when the first or second MDMC pulsed current frequencies applied to the patient have the optimal MDMC pulse frequency for that patient's condition and the optimal MDMC pulse frequency for the tissue type affected thereby. In the case where either or both of the diagnosed or known tissues or condition affected thereby are associated with a plurality of frequencies (e.g., as shown in Tables 1 and 2 above), the practitioner may apply a plurality of combinations of these frequencies in order to identify the optimal MDMC pulse frequencies for treatment of the condition and the tissue type for that specific patient, as described in greater detail with respect to FIG. 5, below. As described herein, the practitioner may determine the optimal MDMC pulse frequency for treatment of the patient's condition and the tissues affected by that condition have been isolated when the patient's muscle situated nearby tissues within the physical manipulation treatment region 131 undergo a rhythmic contraction at a frequency (e.g., below 5 Hz) that is well below either of the applied MDMC pulse frequencies (e.g., above 20 Hz).

If the practitioner observes rhythmic contraction of the patient's muscle within the physical manipulation target region, this may indicate the first applied current frequency is set to the most optimal tissue-type treatment pulse frequency and the second applied current frequency is set to the most optimal condition treatment pulse frequency. The method may then continue to block 524 for identification of the tissue-type treatment pulse frequency of interest as the tissue treatment pulse frequency and identification of the condition treatment pulse frequency of interest as the condition treatment pulse frequency of interest for further treatment during this same physical manipulation treatment session or future sessions. If the practitioner does not observe rhythmic contraction of the patient's muscle within the physical manipulation target region, this may indicate any of the first applied current frequency is not set to the most optimal tissue-type treatment pulse frequency or the second applied current frequency is not set to the most optimal condition treatment pulse frequency or may indicate that the physical manipulation target region is in an incorrect location when the rhythmic contractions may be detected in a different location. The method may then proceed to block 514 to determine whether other frequencies associated with the identified condition have yet to be tested for the rhythmic contractions.

At block 514, in an embodiment in which the patient's muscle within the physical manipulation target region do not undergo rhythmic contraction in response to application of the first and second MDMC pulsed frequencies, the practitioner may determine whether all frequencies for the previously identified condition have been tested. For example, the practitioner in an embodiment in which the identified condition is vertebral spinal pressure and the current condition treatment pulse frequency of interest is 20 Hz, the practitioner may determine that the condition treatment pulse frequency of 11 Hz has not yet been tested to determine whether it will produce rhythmic contractions of muscles nearby the vertebrae so diagnosed. If other frequencies associated with the identified condition have yet to be tested for rhythmic contractions, the method may proceed to block 516 for selection of the next of these untested frequencies as the tissue type or condition treatment pulse frequency of interest to be tested. If none of the frequencies associated with the identified condition, when combined with the first applied current frequency having a first MDMC pulse frequency equivalent to the tissue treatment pulse frequency of interest have produced the rhythmic contractions of the muscle situated nearby the affected vertebrae, this may indicate that the tissue treatment pulse frequency of interest may not be optimal for treatment of this specific patient's tissues affected by the identified condition. The method may then proceed to block 518 to determine whether other frequencies associated with the tissues affected by the identified condition have yet to be tested for rhythmic contractions.

The practitioner may identify the next of a plurality of MDMC pulse frequencies associated with the previously identified condition as the condition treatment pulse frequency of interest in an embodiment at block 516. For example, in an embodiment in which the practitioner determines the 11 Hz condition treatment pulse frequency associated with vertebral spinal pressure has not yet been tested, the practitioner may identify the 11 Hz MDMC pulse frequency associated with vertebral spinal pressure as the condition treatment pulse frequency of interest. The method may then return to block 508 to test whether the new condition treatment pulse frequency of interest (e.g., 11 Hz) produces rhythmic contractions of the patient's muscle within the physical manipulation target region. By repeating the loop between blocks 508 and 516, the practitioner may test whether any of the MDMC pulse frequencies associated with the identified condition (e.g., 20 Hz or 11 Hz associated with vertebral spinal pressure) within Table 1 may be combined with another applied current frequency having the MDMC pulse frequency identified at block 502 as the tissue-type treatment pulse frequency of interest (e.g., 330 Hz associated with vertebral disc tissue) produces the rhythmic contractions of the patient's muscle within the physical manipulation target region. Such rhythmic contraction indicates the optimal MDMC pulse frequency for treatment of the tissue type and the optimal MDMC pulse frequency for treatment of the patient's identified condition affecting that tissue have been isolated. If the practitioner repeats the loop between blocks 508 and 516 for all MDMC pulse frequencies associated with the identified condition, and none of these MDMC pulse frequencies produce rhythmic contractions in combination with the tissue treatment pulse frequency identified at block 502, this may indicate that the tissue treatment pulse frequency identified at block 502 is not the optimal tissue treatment pulse frequency for treatment of the patient's current complaints, and the method may proceed to block 518.

At block 518, the practitioner in an embodiment may determine whether other frequencies for the tissues affected by the identified condition have been tested for rhythmic contraction. For example, the practitioner in an embodiment in which the identified condition is vertebral spinal pressure affecting a vertebral disc tissue type and in which the current tissue treatment pulse frequency of interest is 330 Hz may determine that the tissue treatment pulse frequency of 630 Hz has not yet been tested to determine whether it will produce rhythmic contractions in muscles nearby tissues affected by the identified vertebral spinal pressure. If other frequencies associated with the tissues for a first MDMC pulsed frequency and those associated with the tissue as affected by the identified condition have yet to be tested for rhythmic contractions, the method may proceed to block 520 for selection of the next of these untested frequencies as the tissue treatment pulse frequency of interest to be tested. If none of the frequencies associated with the tissue type in a first MDMC pulsed frequency and affected by an identified condition, when combined with the second applied current frequency having an MDMC pulse frequency equivalent to a condition treatment pulse frequency of interest have produced the rhythmic contractions of muscles within the physical manipulation target region, this may indicate that the previous diagnosis (e.g., identified condition affecting a known tissue type) is not the cause of the patient's current complaints within the physical manipulation target region or that the patient's tissue is not responding to the first or second MDMC pulsed frequency. The method may then proceed to block 522 for determination or possible diagnosis of a currently unknown or undiagnosed condition affecting potentially a plurality of tissues within the physical manipulation target region by practitioner. If a determination is reached, the method may begin again at block 502 for the newly identified condition.

The practitioner may identify the next of a plurality of MDMC pulse frequencies associated with the tissues affected by the previously determined condition as the tissue treatment pulse frequency of interest in an embodiment at block 520. For example, in an embodiment in which the practitioner determines the 630 Hz condition treatment pulse frequency associated with vertebral disc tissue has not yet been tested, the practitioner may identify the 630 Hz MDMC pulse frequency associated with vertebral disc tissue as the tissue treatment pulse frequency of interest. The method may then return to block 504 to test whether the new tissue treatment pulse frequency of interest produces rhythmic contractions of the patient's muscle within the physical manipulation target region. By repeating the loop between blocks 504 and 520, the practitioner may test whether any of the second MDMC pulse frequencies of block 506 associated with the identified condition within Table 1 may be combined with any of the MDMC pulse frequencies associated in Table 2 with the tissue type affected by the identified condition may produce the rhythmic contractions of the patient's muscle within the physical manipulation target region. Such rhythmic contraction at block indicates the optimal MDMC pulse frequency for treatment of the patient's identified condition and the optimal MDMC pulse frequency for treatment of the tissue type affected by that identified condition have been isolated.

As described above, with respect to block 518, if none of the frequencies associated with the tissue type affected by the identified condition, when combined with the first applied current frequency having an MDMC pulse frequency equivalent to the condition treatment pulse frequency of interest have produced the rhythmic contractions, this may indicate that the previous diagnosis (e.g., identified condition affecting a known tissue type) is not the cause of the patient's current complaints within the physical manipulation target region. The method may then proceed to block 522 for possible diagnosis of a currently unknown or undiagnosed condition affecting potentially a plurality of tissues.

At block 522, in an embodiment in which none of the combinations of MDMC pulse frequencies associated with the identified condition and MDMC pulse frequencies associated with the tissue type affected by the identified condition produce the rhythmic contraction of the patient's muscle within the physical manipulation target region, the practitioner may test for or consider other possible diagnoses. For example, a patient may have received a diagnosis of vertebral spinal pressure affecting vertebral disc tissue. If no combination of known tissue treatment pulse frequencies and condition treatment pulse frequencies cause rhythmic contractions of muscles nearby the identified vertebrae at block 512, this may indicate another condition is affecting the spinal disc tissue, or that the spinal pressure is affecting another tissue type to produce the patient's current complaint. In such a scenario, the method may end for the previous diagnosis and the method may restart at block 502 for a new diagnosis made by the practitioner. For example, the spinal disc tissue may be torn or herniated—a physical condition associated with different condition treatment pulse frequencies (e.g., 124 Hz as shown in Table 1) than the identified condition of vertebral spinal pressure. As another example, the vertebral spinal pressure may be affecting nearby nerves, rather than the vertebral disc. These nerves in such an example embodiment may be associated with different tissue treatment pulse frequencies (e.g., 396 Hz as shown in Table 2) than the vertebral disc tissue type described above. The practitioner in an embodiment may attempt to identify a condition other than the previously identified condition or tissue type other than the tissue type previously identified as affected by the previously identified condition in such circumstances, as described in greater detail below with respect to FIG. 6. The method for determining a tissue treatment pulse frequency and a condition treatment pulse frequency for MDMC pulsed frequencies to apply to a patient during electrotherapy treatment of a identified condition and tissue may then end.

Upon observing rhythmic contractions of the patient's muscle within the physical manipulation target region in an embodiment, the practitioner at block 524 may identify the tissue treatment pulse frequency of interest as the tissue treatment pulse frequency and the condition treatment pulse frequency of interest as the condition treatment pulse frequency. For example, in an embodiment, the patient may have been previously identified with vertebral spinal pressure affecting vertebral disc tissue. The practitioner in such an embodiment may observe rhythmic contractions of muscles situated nearby the affected vertebral discs and within the physical manipulation target region at block 512 upon application at block 510 of a first MDMC pulsed frequency having an MDMC pulse frequency equivalent to the tissue treatment pulse frequency of 330 Hz, and application of a second MDMC pulsed frequency having an MDMC pulse frequency equivalent to the condition treatment pulse frequency of 20 Hz. In such an embodiment, the practitioner may identify the tissue treatment pulse frequency for optimal treatment of the patient's tissues affected by the identified condition as 330 Hz. The practitioner in such an embodiment may also identify the condition treatment pulse frequency for optimal treatment of the identified condition as 20 Hz. The method for determining a tissue treatment pulse frequency and a condition treatment pulse frequency for applied current frequencies to apply to a patient during treatment of a identified condition and tissue may then end

FIG. 6 is a flow diagram illustrating a method of diagnosing a patient's tissue for treatment and physical condition and using the mixed direct macro-current (MDMC) electrotherapy system according to an embodiment of the present disclosure. As described above with respect to FIG. 5, in some situations in which a patient has received a previous diagnosis affecting a known tissue type, none of the combinations of MDMC pulse frequencies associated with the identified condition and MDMC pulse frequencies associated with the tissue type affected by the identified condition produce the rhythmic contraction of the patient's muscle within the physical manipulation target region. This may indicate that a physical condition other than the previously identified condition may be causing the patient's current discomfort. This may alternatively indicate that the identified condition is affecting a previously unknown tissue type. In other situations, the patient may have not yet received a diagnosis, and presents simply with symptoms or complaints the practitioner may use to infer possible diagnoses. In each of these scenarios, the practitioner in an embodiment may use the method of FIG. 6 to identify the patient's physical condition underlying current discomfort and the tissues affected thereby.

At block 602, the practitioner in an embodiment may identify the first of a plurality of tissue types located within the patient's physical manipulation target region as a tissue type of interest. As described above with respect to FIG. 1, in some embodiments, the practitioner may use the MDMC electrotherapy system 100 to identify an unknown condition affecting potentially several tissues not yet specifically identified within the patient's physical manipulation target region 131. As described herein, the combined application of a first MDMC pulsed current frequency targeted toward treatment of a patient's affected tissues and application of a second MDMC pulsed current frequency targeted toward treatment of an existing but undiagnosed condition within the patient's physical manipulation target region 131 may produce rhythmic contraction of the patient's 130 tissues within the physical manipulation target region 131. This rhythmic contraction may occur at a frequency (e.g., below 5 Hz) below the MDMC pulse frequencies (e.g., above 10 Hz) of either the first or second MDMC pulsed frequencies so applied to the physical manipulation target region 131. Observance of these rhythmic contractions in an embodiment may indicate that the practitioner has isolated the first MDMC pulsed frequency optimal for treatment of the patient's tissues and the second MDMC pulse frequency optimal for treatment of the patient's condition affecting those tissues.

For example, a patient may complain of pain or discomfort surrounding a major joint such as the knee. In such an embodiment, if the patient has not yet received any diagnosis explaining this pain or discomfort, the practitioner at block 602 may identify one or several tissue types within and surrounding the knee joint (e.g., bone, ligament, tendon, bursa, cartilage, veins) as the tissue type of interest. For example, the practitioner at block 602 may identify bone as the tissue type of interest. In another example, the practitioner may identify cartilage as the tissue type of interest.

In an embodiment in which the patient has received a previous diagnosis that did not respond to electrotherapy targeted toward the previously identified condition or tissues known to be affected thereby, the practitioner may identify one of a plurality of tissues surrounding the tissues already known to be affected by the identified condition as the tissue type of interest. In another example, a patient may have received a diagnosis of vertebral spinal pressure affecting vertebral disc tissue. If no combination of tissue treatment MDMC pulse frequencies or condition treatment MDMC pulse frequencies cause rhythmic contractions of muscles nearby the identified vertebrae or within the physical manipulation target region (e.g., as determined at block 512 of FIG. 5), this may indicate another condition is affecting the spinal disc tissue, or that the spinal pressure is affecting another tissue type to produce the patient's current complaint. For example, the vertebral spinal pressure may be affecting nearby nerves, rather than the vertebral disc. These nerves in such an example embodiment may be associated with different tissue treatment pulse frequencies (e.g., 396 Hz as shown in Table 2) than the vertebral disc tissue type (e.g., associated with 330 Hz, 630 Hz, and 710 Hz, as shown in Table 2). In such an embodiment, the practitioner at block 602 may identify nerve tissue as the tissue type of interest, in order to test whether the previously identified condition is affecting nerve tissue rather than the vertebral disc tissue previously known to be affected by the previously identified vertebral spinal pressure condition. In another example of such an embodiment, the practitioner at block 602 may identify vertebral disc tissue as the tissue type of interest, in order to test whether the vertebral disc tissue already known to be affected by the previously identified condition of vertebral spinal pressure is also being affected by another, currently unknown physical condition to cause the patient's primary complaints.

The practitioner at block 604 may identify the first of a plurality of conditions potentially capable of causing a patient's symptoms as a condition of interest in an embodiment. For example, a patient may complain of pain or discomfort surrounding a major joint such as the knee. In such an embodiment, if the patient has not yet received any diagnosis explaining this pain or discomfort, the practitioner at block 604 may identify one or several physical conditions (e.g., tear, fracture, blood clot, inflammation, fibrosis or scarring, cysts, nerve pain, trauma, tissue degeneration or necrosis) that could be affecting tissue types within and surrounding the knee joint as the condition of interest. For example, the practitioner at block 604 may identify fracture as the condition of interest. In another example, the practitioner may identify trauma as the condition of interest.

In an embodiment in which the patient has received a previous diagnosis that did not respond to electrotherapy targeted toward the previously identified condition or tissues known to be affected thereby, the practitioner may identify one of a plurality of physical conditions other than the identified condition that could affect the tissues already known to be affected by the identified condition as the condition of interest. For example, a patient may have received a diagnosis of vertebral spinal pressure affecting vertebral disc tissue. If no combination of known tissue treatment first MDMC pulse frequencies and condition treatment second MDMC pulse frequencies cause rhythmic contractions of muscles nearby the identified vertebrae or within the physical manipulation target region (e.g., as determined at block 512 of FIG. 5), this may indicate another condition is affecting the spinal disc tissue, or that the spinal pressure is affecting another tissue type to produce the patient's current complaint. For example, the spinal disc tissue may be torn or herniated—a physical condition associated with different condition treatment pulse frequencies (e.g., 124 Hz as shown in Table 1) than the identified condition of vertebral spinal pressure (e.g., associated with 20 Hz, and 11 Hz in Table 1). In such an embodiment, the practitioner may identify a tear or herniation as the physical condition of interest at block 604, in order to test whether the vertebral disc tissue already known to be affected by the previously identified condition of vertebral spinal pressure is also being affected by the additional physical condition of tearing or herniation to cause the patient's primary complaints. In another example of such an embodiment, the practitioner may identify vertebral spinal pressure as the condition of interest, in order to test whether the previously identified condition is affecting tissues other than the tissue (e.g., vertebral disc tissue) previously identified as being affected by that physical condition.

At block 606, the practitioner in an embodiment may determine whether any of the first or second MDMC pulsed frequencies or a combination of MDMC pulsed frequencies for the tissue type of interest and the condition of interest causes rhythmic contraction of the patient's muscle within the physical manipulation target region. The practitioner in an embodiment may employ the method described above with respect to FIG. 5 to make such a determination, for example. In the case where a patient complains of pain or discomfort in a knee but has yet to receive any diagnosis, for example, the practitioner may test whether one of a plurality of physical conditions possibly affecting one of a plurality of tissue types in and around the knee may be causing the patient's physical discomfort or pain. For example, the practitioner may employ the method described above with respect to FIG. 5 determine whether any combination of tissue treatment pulse frequencies associated with bone tissue and condition treatment pulse frequencies associated with fractures causes rhythmic contraction of the patient's muscle nearby the bone within the physical manipulation target region. As another example, the practitioner may employ the method described above with respect to FIG. 5 to determine whether any combination of tissue treatment pulse frequencies associated with cartilage and condition treatment pulse frequencies associated with tears causes rhythmic contraction of the patient's muscles nearby the cartilage within the physical manipulation target region.

In another specific example, the practitioner may determine whether any combination of tissue treatment pulse frequencies associated with nerve tissue and condition treatment pulse frequencies associated with vertebral discs causes rhythmic contraction of the patient's muscle within the physical manipulation target region. In yet another example, the practitioner may determine whether any combination of tissue treatment pulse frequencies associated with vertebral disc tissue and condition treatment pulse frequencies associated with tears or herniation causes rhythmic contraction of the patient's muscle within the physical manipulation target region.

If at block 606, the application to the patient within the physical manipulation target region of a first MDMC pulsed frequency having a tissue treatment pulse frequency associated with the tissue type of interest and a second MDMC pulsed frequency having a condition treatment pulse frequency associated with the physical condition of interest produces rhythmic contraction of the patient's muscle within the physical manipulation target region at a frequency lower than one or both of the tissue treatment pulse frequency or the condition treatment pulse frequency, the method may proceed to block 608. This may indicate that the combination of the first and second applied current frequency MDMC pulse frequencies is effective for electrotherapy treatment of the physical condition affecting the patient's tissue within the physical manipulation target region.

If at block 606 none of the available combinations of condition treatment pulse frequencies or tissue treatment pulse frequencies associated with the condition of interest and the tissue type of interest, respectively, produces the rhythmic contraction of the patient's muscle within the physical manipulation target region, this may indicate that the condition of interest and the tissue of interest are not the cause of the patient's symptoms within the physical manipulation target region. The method may then proceed to block 610 to investigate whether another condition may be affecting the tissue type of interest to produce the patient's symptoms within the physical manipulation target region.

Returning to block 608, in an embodiment in which application of a first MDMC pulsed frequency having a tissue treatment pulse frequency associated with the tissue type of interest and a second MDMC pulsed frequency having a condition treatment pulse frequency associated with the physical condition of interest produces rhythmic contraction of the patient's muscle within the physical manipulation target region, the practitioner may identify the tissue type of interest as the tissue type in need of treatment and the condition of interest as the condition to be treated at block 608. Such rhythmic contraction at a frequency below one or both of the frequencies associated with the condition of interest and the tissue type of interest in an embodiment may indicate the condition of interest is affecting the tissue type of interest to produce the patient's symptoms within the patient's physical manipulation target region. It may also assist with location for treatment within the target physical manipulation area.

In an embodiment, the practitioner may observe rhythmic contractions of the patient's muscle within the physical manipulation target region having a frequency (e.g., under 5 Hz) that is less than either the tissue treatment pulse frequency associated with the tissue type of interest, or the condition treatment pulse frequency associated with the condition of interest. For example, in the case where a patient complains of pain or discomfort in a knee but has yet to receive any diagnosis, the practitioner may observe rhythmic contraction at a frequency under 5 Hz in response to application of a first MDMC pulsed current frequency having an MDMC pulse frequency equivalent to the tissue treatment pulse frequency of 59 Hz (e.g., associated with bone tissue in Table 2) and a second MDMC pulsed current frequency having an MDMC pulse frequency equivalent to the condition treatment pulse frequency of 124 Hz (e.g., associated with fracture or break in Table 1). In such an example embodiment, the practitioner may determine that a bone fracture is the source of the patient's current complaint, and identify the bone as the tissue type in need of treatment and fracture as the condition to be treated.

As another example, the practitioner may observe rhythmic contraction at a frequency under 5 Hz in response to application of a first MDMC pulsed current frequency having an MDMC pulse frequency equivalent to the tissue treatment pulse frequency of 157 Hz (e.g., associated with cartilage tissue in Table 2) and a second MDMC pulsed current frequency having an MDMC pulse frequency equivalent to the condition treatment pulse frequency of 124 Hz (e.g., associated with tearing in Table 1). In such an example embodiment, the practitioner may determine that a cartilage tear is the source of the patient's current complaint, and identify the cartilage as the tissue type in need of treatment and tearing as the condition to be treated.

As another example, the practitioner may observe rhythmic contractions under 5 Hz in response to application of a first MDMC pulsed current frequency having an MDMC pulse frequency equivalent to the tissue treatment pulse frequency of 396 Hz (e.g., associated with peripheral nerve tissue in Table 2) and a second MDMC pulsed current frequency having an MDMC pulse frequency equivalent to the condition treatment pulse frequency of 20 Hz (e.g., associated with vertebral spinal pressure in Table 1). In such an example embodiment, the practitioner may determine that nerve tissue affected by the identified condition of vertebral spinal pressure is the source of the patient's current complaint, as opposed to the vertebral disc previously known to be affected by the vertebral spinal pressure condition. The practitioner in such an embodiment may then identify the nerve tissue as the tissue type in need of treatment and vertebral spinal pressure as the condition to be treated.

As yet another example, the practitioner may observe rhythmic contractions under 5 Hz in response to application of a first MDMC pulsed current frequency having an MDMC pulse frequency equivalent to the tissue treatment pulse frequency of 330 Hz (e.g., associated with vertebral disc tissue in Table 2) and a second MDMC pulsed current frequency having an MDMC pulse frequency equivalent to the condition treatment pulse frequency of 124 Hz (e.g., associated with tearing in Table 1). In such an example embodiment, the practitioner may determine that the herniation of tearing of the vertebral disc tissue is causing the patient's current complaint, as opposed to the previously identified vertebral spinal pressure applied to the vertebral disc. The practitioner in such an embodiment may then identify the vertebral disc tissue as the tissue type in need of treatment and tearing or herniation as the condition to be treated.

The practitioner may thus use the mixed direct macro-current (MDMC) electrotherapy system to diagnose the physical condition that is affecting a specific tissue type to produce the patient's symptoms within the physical manipulation target region. The method for diagnosing the patient's specific condition and the tissue type affected by that condition may then end.

Returning to block 610, in an embodiment in which none of the available combinations of frequencies associated with the tissue type of interest or the condition of interest produce the rhythmic contraction of the patient's muscle within the physical manipulation target region, the practitioner may determine whether each of the possible conditions capable of affecting the tissue type of interest within the physical manipulation target region have been tested using the MDMC electrotherapy system at block 610. For example, the practitioner in an embodiment may determine that no combination of tissue treatment pulse frequencies associated with bone tissue and condition treatment pulse frequencies associated with fractures causes rhythmic contraction of the patient's tissues. In such an example embodiment, the practitioner at block 610 may determine whether all condition treatment pulse frequencies associated with all physical conditions that could be affecting the patient's bone tissue within the knee have been tested using the method described above with respect to block 606 and FIG. 5.

If each of the possible conditions capable of affecting the tissue type of interest within the physical manipulation target region have not yet been tested at block 610, the method may proceed to block 612 for testing of other possible conditions that may affect the tissue type of interest. If the second MDMC pulsed frequencies associated with each of the possible conditions capable of affecting the tissue type of interest have been tested at block 610 however, this may indicate that the tissue type of interest is not the tissue type causing the patient's symptoms within the physical manipulation target region. The method may then proceed to block 614 to test other tissue types within the patient's physical manipulation target region.

Returning to block 612, in an embodiment in which each of the possible conditions capable of affecting the tissue type of interest have not yet been tested, and none of the previous combinations of frequencies for tissue types and conditions have produced rhythmic contraction in the physical manipulation target region, the practitioner may identify the next of a plurality of untested physical conditions as the condition of interest. For example, in an embodiment in which the condition treatment pulse frequencies associated with fracture in combination with the tissue treatment pulse frequency of bone failed to produce the rhythmic contractions of muscles within the physical manipulation target region, the practitioner may identify trauma as the next condition of interest. Trauma may include injuries less severe than fracture, such as contusions, bruising, or internal bleeding. The method may then proceed back to block 606 to determine (e.g., through use of the method described herein or with respect to FIG. 5 above) whether the patient's complaints could be caused by, for example, a deep bone bruise, rather than a bone fracture. By repeating the loop between block 612 and 606 in an embodiment, the practitioner may thus use the mixed direct macro-current (MDMC) electrotherapy system to identify the physical condition that is affecting a specific tissue type to produce the patient's symptoms within the physical manipulation target region to identify a second MDMC pulsed frequency for a particular condition.

In an embodiment in which each of the possible conditions capable of affecting the tissue type of interest have been tested, the practitioner may determine at block 614 whether each of the tissue types within the patient's physical manipulation target region have been tested. For example, the practitioner in an embodiment may determine that no combination of tissue treatment pulse frequencies associated with bone tissue and condition treatment pulse frequencies associated with fractures or tears causes rhythmic contraction of the patient's tissues. As shown in Table 1, both fractures (e.g., “breaks”) and tears are associated with the same condition treatment pulse frequency (e.g., 124 Hz). In such an example embodiment, the practitioner at block 614 may determine whether all tissue treatment pulse frequencies associated with all tissue types that could be affected by a tear or fracture within the knee have been tested using the method described above with respect to block 606 and FIG. 5.

If each of the tissue types within the patient's physical manipulation target region have not yet been tested at block 614 using the MDMC electrotherapy system, this may indicate that the tissue type of interest is not the source of the patient's symptoms within the physical manipulation target region. The method may then proceed to block 616 for testing of another tissue type within the patient's physical manipulation target region using the MDMC electrotherapy system. If each of the tissue types within the patient's physical manipulation target region have been tested at block 614, the method may proceed to block 618 for a generalized treatment approach that does not target a specific combination of an identified condition affecting a known tissue type.

At block 616, in an embodiment in which each of the tissue types within the patient's physical manipulation target region have not yet been tested, and none of the previously tested combinations of frequencies for tissue types and conditions have produced rhythmic contraction in the physical manipulation target region, the practitioner may identify the next of a plurality of untested tissue types within the physical manipulation target region as the tissue type of interest. For example, in an embodiment in which the condition treatment pulse frequencies associated with fracture or tear in combination with the tissue treatment pulse frequency of bone failed to produce the rhythmic contractions of muscle within the physical manipulation target region, the practitioner may identify cartilage as the next tissue type of interest. The method may then proceed back to block 606 to determine (e.g., through use of the method described with respect to FIG. 5 above) whether the patient's complaints could be caused by, for example, a torn cartilage, rather than a bone fracture. By repeating the loop between block 616 and 606 in an embodiment, the practitioner may thus use the MDMC electrotherapy system to diagnose or identify the physical condition that is affecting a specific tissue type to produce the patient's symptoms within the physical manipulation target region. Once a tissue type is identified, a loop between blocks 612 and 606 may identify an associated condition affecting that tissue type as described in embodiments herein.

At block 618, in an embodiment in which testing of all known combinations of possible conditions affecting possible tissue types within the physical manipulation target region has not produced the rhythmic contractions of the patient's tissues within the physical manipulation target region, the practitioner may adopt a generalized treatment approach in an optional embodiment that does not target a specific combination of an identified condition affecting a known tissue type. Such a generalized treatment approach may, for example, apply a first MDMC pulsed current frequency having a condition treatment pulse frequency associated with generalized treatment (e.g., 294 Hz, 321 Hz, or 9 Hs) shown in Table 1 and serially apply one of several different MDMC pulsed frequencies, each having a tissue treatment pulse frequency associated with different tissues within and surrounding the patient's physical manipulation target region (e.g., knee tissues such as bone, cartilage, tendon, ligament, vein, nerve). In such a way, the practitioner may globally address any suspected conditions of tissues known to be within the patient's physical manipulation target region to the best of her abilities. The method for diagnosing a patient's physical condition and tissue for treatment using the MDMC electrotherapy system may then end.

FIG. 7 is a flow diagram illustrating a method of treating a patient's identified condition of a tissue known to need treatment through electrotherapy-assisted physical manipulation according to an embodiment of the present disclosure. As described herein, the practitioner may perform electrotherapy-assisted physical manipulation treatment of the patient's musculature or soft tissue within a physical manipulation target region in order to simultaneously provide physical manipulation therapy and promote healing of tissues known to be affected by an identified physical condition. Upon identification of the optimal MDMC pulse frequency for the patient's tissue type and identification of the optimal MDMC pulse frequency for the patient's identified condition affecting that tissue in an embodiment (e.g., as described above with respect to FIGS. 5 and 6), the practitioner may initiate an electrotherapy-assisted physical manipulation treatment session. In such a session, the practitioner may apply a first MDMC pulsed current frequency targeted toward treatment of a tissue and a second MDMC pulsed current frequency targeted toward treatment of a patient's identified condition affecting that tissue via a pair of electrodes to the patient's skin within a physical manipulation target region.

At block 702, one or more stationary electrodes may be placed on the patient nearby a physical manipulation target region identified by the practitioner or patient in an embodiment. For example, in an embodiment described with respect to FIG. 1, a mixed direct macro-current (MDMC) electrotherapy pulsed frequency generator 110 in an embodiment may transmit a first MDMC applied current frequency via a pair of electrodes that may be both static or one static and one mobile, such as mobile electrode 123 and static electrode 113 situated within a physical manipulation target region 131 or treatment area of a patient's musculature via an electrically conductive cable or wire 112. In other embodiments, at least one static electrode may be placed anywhere on the patient's body, not necessarily within the physical manipulation target region 131.

In an embodiment in which the mobile electrode comprises an electrotherapy glove, the practitioner's hand may be inserted within an electrotherapy glove having an electroconductive exterior surface at block 704. For example, as described in an embodiment with respect to FIG. 1, the first MDMC pulsed current frequency in an embodiment may be applied to a patient during physical manipulation of a patient's musculature or soft tissues (e.g., physical manipulation therapy) by a practitioner via an electrotherapy glove 120 with a mobile electrode 123 worn by the practitioner 122, or during diagnosis of a physical condition of a patient's tissue to be treated. One or more mobile electrodes 123 and 121 may be placed along the exterior of the electrotherapy glove at positions relative to the practitioner's hand commonly used in various physical manipulation techniques.

At block 706, the electrotherapy glove or other type of mobile electrode, such as a physical manipulation tool used by the practitioner, in an embodiment may be operably connected to the mixed direct macro-current (MDMC) electrotherapy system in an embodiment. For example, in an embodiment described with respect to FIG. 1, the mixed direct macro-current (MDMC) electrotherapy pulsed frequency generator 110 may also transmit a second MDMC pulsed current frequency to second mobile electrode 121 of an electrotherapy glove 120 worn by a practitioner 122 to conduct physical manipulation and electrotherapy and a paired static electrode 115, via an electrically conductive cable or wire 111.

A pre-diagnostic applied current frequency may be transmitted from the MDMC electrotherapy system to the electrode pair, such as a paired mobile electrode manipulated by the practitioner and a static electrode, in an embodiment at block 708. For example, the practitioner 122 may use the mobile electrode and static electrode pairs (e.g., 113 and 123 or 115 and 121) to identify a physical condition of a patient's tissue to be treated by beginning with a patient 130 identifying a physical manipulation target region 131 that is causing the patient 130 discomfort or pain according to various embodiments herein. In an embodiment, the practitioner 122 may apply the first MDMC pulsed frequency, the second MDMC pulsed frequency, or a combination within the physical manipulation target region 131 (or anywhere on the patient's body) via one or more electrode pairs including the electrotherapy glove, and initially instruct the MDMC electrotherapy pulsed frequency generator 110 to produce a pre-diagnostic applied current frequency having an amplitude (e.g., at or below one Volt) relatively lower than that routinely used during therapeutic physical manipulation in an example embodiment before increasing amplitude levels for additional electrotherapy.

At block 710, the practitioner may apply the pre-diagnostic applied current frequency to the area of complaint on the patient, via the electrotherapy glove or other type of mobile electrode and paired with a second electrode. For example, in an embodiment described with reference to FIG. 1, the practitioner 122 may apply the pre-diagnostic applied current frequency to the patient's physical manipulation target region 131 via contact between the mobile electrode 120 and a portion of the patient's skin within the physical manipulation target region 131, causing the patient's musculature or soft tissue to conduct the pre-diagnostic applied current frequency between the mobile electrode and the stationary electrode pair or pairs for the first MDMC pulsed frequency, the second MDMC pulsed frequency, or a combination.

The practitioner may increase the amplitude of the pre-diagnostic applied current frequency in an embodiment at block 712 until the patient reports or the practitioner observes muscle contraction within the physical manipulation target region to identify a diagnostic amplitude for an applied current frequency used to identify a physical condition of a patient's tissue to be treated. As described in an embodiment with respect to FIG. 2, for example, a knob, LCD touchscreen input, or other input device 203 in an embodiment may be used in order to adjust an amplitude or a frequency of either or both of the first or second MDMC pulsed current frequency, including the pre-diagnostic applied current frequency. As also described in an example embodiment with respect to FIG. 1, the practitioner 122 may use such a knob to slowly increase the amplitude (e.g., voltage) of the applied, pulsed current frequency or frequencies until the patient reports, or the practitioner observes a contraction of the patient's musculature between or surrounding the mobile electrode paired with the stationary electrode in one or more electrode pairs. This observation may indicate an amplitude (e.g. voltage) the practitioner may use to instruct the MDMC electrotherapy pulsed frequency generator 110 to generate a one or more diagnostic applied current frequencies such as the first MDMC pulsed frequency, the second MDMC pulsed frequency, or a combination that the practitioner 122 may use to more specifically identify an optimal MDMC pulse frequency for a first MDMC pulsed current frequency targeted toward treatment of a tissue and an optimal MDMC pulse frequency for a second MDMC pulsed current frequency targeted toward treatment of an identified condition affecting that tissue to cause the patient's discomfort or pain within the physical manipulation target region.

At block 714, the practitioner may identify the patient's a tissue type and physical condition located within the physical manipulation target region affected thereby in an embodiment. For example, as described in greater detail above with respect to FIG. 6, the practitioner may use the MDMC electrotherapy system including the mixed direct macro-current (MDMC) electrotherapy pulsed frequency generator, the stationary electrode, and the mobile electrode to identify the patient's physical condition and a tissue affected thereby in an embodiment.

The practitioner in an embodiment may identify a tissue treatment pulse frequency and a condition treatment pulse frequency to be applied to a patient's skin during an electrotherapy-assisted physical manipulation treatment at block 716. Upon diagnosis of a patient's physical condition and tissues affected thereby, the practitioner in an embodiment may identify one of a plurality of frequencies known to promote healing of such physical conditions or tissues affected thereby, as described in greater detail above with respect to FIG. 5. As described herein, such diagnostic methods may be performed in the same session as a physical manipulation treatment due to the relatively high DMC voltage and current of the applied current frequencies applied to the patient in comparison to previous systems employing lower-voltage AC applied current frequencies and DC microcurrents (e.g., below one milliampere (mA)).

At block 718, the practitioner in an embodiment may set channel A of the MDMC electrotherapy pulsed frequency generator to transmit to the stationary electrode a first applied current frequency having a first MDMC pulsed frequency equivalent to a tissue treatment pulse frequency. For example, such a tissue treatment pulse frequency may be identified at block 716 above and described in greater detail above with respect to FIG. 5. The tissue treatment pulse frequency may be tailored to promote healing of a specifically identified tissue within the patient's physical manipulation target region affected by the condition identified at block 714. For example, in an embodiment described with reference to FIG. 1, the practitioner in an embodiment may set the MDMC pulse frequency for the first MDMC pulsed frequency applied to the patient 130 via a first electrode pair including an electrotherapy glove to be equivalent to the tissue treatment pulse frequency associated in Table 2 with the tissue type identified at block 714 (e.g., the vertebral disc tissue type). More specifically, the practitioner may set the MDMC pulse frequency for a first MDMC pulsed current frequency output on Channel A of the MDMC electrotherapy pulsed frequency generator 110 to a value of 330 Hz.

The practitioner in an embodiment may set channel B of the MDMC electrotherapy pulsed frequency generator to transmit to the mobile electrode a second applied current frequency having a second MDMC pulsed frequency equivalent to a condition treatment pulse frequency at block 720. For example, such a condition treatment pulse frequency may be identified at block 716 above and described in greater detail above with respect to FIG. 5. The condition treatment pulse frequency may be tailored to heal a condition identified at block 714 and affecting a tissue within the patient's physical manipulation target region. For example, in an embodiment described with reference to FIG. 1, the practitioner in an embodiment may set the MDMC pulse frequency for a second MDMC pulsed current frequency applied to the patient 130 via a second electrode pair, including an electrode therapy glove to be equivalent to the condition treatment pulse frequency associated in Table 1 with the physical condition identified at block 714 (e.g., vertebral spinal pressure). More specifically, the practitioner may set the MDMC pulse frequency for a second MDMC pulsed current frequency output on Channel B of the MDMC electrotherapy pulsed frequency generator 110 to a value of 20 Hz.

At block 722, the practitioner in an embodiment may treat the patient's tissue within the physical manipulation target region with an electrotherapy-assisted physical manipulation treatment to treat the identified physical condition affecting the patient's tissues. For example, the practitioner may sweep the electrotherapy glove 120 across the patient's skin within the physical manipulation target region or may conduct physical manipulation of the physical manipulation target region. In an embodiment where the electrode pair comprises a mobile electrode in an electrotherapy glove, the practitioner may further perform manual manipulation of the patient's tissues within the physical manipulation target region using the hand inserted within the electrotherapy glove. Such physical manipulation of the patient's tissues or skin may allow the practitioner to deliver, via the mobile electrode and the electrotherapy glove 120, a first MDMC pulsed current frequency targeted toward treatment of the patient's tissues, and apply another second MDMC pulsed current frequency targeted toward treatment of the patient's identified condition affecting the tissue. The method for treating an identified condition of a patient's tissue needing treatment using an MDMC electrotherapy system in an embodiment may then end.

The invention has broad applicability and can provide many benefits as described and shown in the examples above. The embodiments will vary greatly depending upon the specific application, and not every embodiment will provide all of the benefits and meet all of the objectives that are achievable by the invention.

Further, whenever the terms “automatic,” “automated,” or similar terms are used herein, those terms will be understood to include manual initiation of the automatic or automated process or step.

In the following discussion and in the claims, the terms “including” and “comprising” are used in an open-ended fashion, and thus should be interpreted to mean “including, but not limited to . . . .” The term “integrated circuit” refers to a set of electronic components and their interconnections (internal electrical circuit elements, collectively) that are patterned on the surface of a microchip.

To the extent that any term is not specially defined in this specification, the intent is that the term is to be given its plain and ordinary meaning. The accompanying drawings are intended to aid in understanding the present invention and, unless otherwise indicated, are not drawn to scale.

Although the present invention and its advantages have been described in detail, it should be understood that various changes, substitutions, and alterations can be made to the embodiments described herein without departing from the spirit and scope of the invention as defined by the appended claims. Moreover, the scope of the present application is not intended to be limited to the particular embodiments of the process, machine, manufacture, composition of matter, means, methods, and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A mixed direct macro-current (MDMC) electrotherapy system comprising:
a stationary electrode in contact with a patient's skin within a physical manipulation target region of a patient's body;
a first electrode for application to the patient's skin by a practitioner during an electrotherapy-assisted physical manipulation treatment an identified tissue type, as determined based on the practitioner's observance of or the patient's reporting of the rhythmic contraction of the patient's muscle;
an MDMC electrotherapy pulsed frequency generator operably connected to the mobile electrode and the stationary electrode to deliver a first MDMC pulsed current frequency having a tissue treatment pulse frequency corresponding to the identified tissue type to the patient's skin, where the first MDMC pulsed current frequency is mixed with an alternating current (AC) current frequency component; and the first MDMC pulsed current frequency causing rhythmic contraction of the patient's muscle within the physical manipulation target region at a contraction pulse frequency that is lower than the tissue treatment pulse frequency, wherein the first MDMC pulsed current frequency has a direct macro-current portion equal to or greater than one milliampere (mA).

2. The MDMC electrotherapy system of claim 1 further comprising:
the MDMC electrotherapy pulsed frequency generator operably connected to a second electrode and a second stationary electrode via a second electrically conductive interface to deliver a second MDMC pulsed current frequency having a condition treatment pulse frequency corresponding to an associated condition of the identified tissue type to the patient's skin.

3. The MDMC electrotherapy system of claim 1, wherein the tissue type to be treated is identified by the practitioner as a type of tissue located within the physical manipulation target region, and the tissue treatment pulse frequency is one of a plurality of frequencies associated with the tissue type to be treated.

4. The MDMC electrotherapy system of claim 2, wherein the identified condition is identified by the practitioner as a condition capable of causing one or more symptoms within the physical manipulation target region, as reported by the patient, and the condition treatment pulse frequency is one of a plurality of frequencies associated with the identified condition.

5. The MDMC electrotherapy system of claim 2, wherein the second MDMC pulsed current frequency causes rhythmic contraction of the patient's muscle within the physical manipulation target region at the contraction pulse frequency that is lower than the condition treatment pulse frequency.

6. The MDMC electrotherapy system of claim 1, wherein the contraction pulse frequency is less than 5 Hz and the tissue treatment pulse frequency is equal to or greater than 10 Hz.

7. The MDMC electrotherapy system of claim 1, wherein the first electrode is a mobile electrode that is on an electrotherapy glove for contacting the patient's skin within the physical manipulation target region during the electrotherapy-assisted physical manipulation treatment.

8. A mixed direct macro-current (MDMC) electrotherapy system comprising:
a stationary electrode in contact with a patient's skin within a physical manipulation target region of a patient's body;
a mobile electrode for application to the patient's skin by a practitioner during an electrotherapy-assisted physical manipulation treatment of an identified condition affecting an identified tissue type, as determined based on the practitioner's observance of or the patient's reporting of the rhythmic contraction of the patient's muscle;
an MDMC electrotherapy pulsed frequency generator operably connected to the mobile electrode via an electrically conductive interface to deliver a first MDMC pulsed current frequency having a condition treatment pulse frequency corresponding to the identified condition to the patient's skin, where the first MDMC pulsed current frequency is mixed with an alternating current (AC) current frequency component; and
the first MDMC pulsed current frequency causing rhythmic contraction of the patient's muscle within the physical manipulation target region at a contraction pulse frequency that is lower than the condition treatment pulse frequency, where the first MDMC pulsed current frequency has a direct macro-current portion equal to or greater than one milliampere (mA).

9. The MDMC electrotherapy system of claim 8 further comprising:
the MDMC electrotherapy pulsed frequency generator operably connected to a second mobile electrode and a second stationary electrode via a second electrically conductive interface to deliver a second MDMC pulsed current frequency having a tissue treatment pulse frequency corresponding to the identified tissue type to the patient's skin.

10. The MDMC electrotherapy system of claim 8, wherein the tissue type to be treated is identified by the practitioner as a type of tissue located within the physical manipulation target region, and the tissue treatment pulse frequency is one of a plurality of frequencies associated with the tissue type to be treated.

11. The MDMC electrotherapy system of claim 8, wherein the identified condition is identified by the practitioner as a condition capable of causing one or more symptoms within the physical manipulation target region, as reported by the patient, and the condition treatment pulse frequency is one of a plurality of frequencies associated with the identified condition.

12. The MDMC electrotherapy system of claim 9, wherein a combination of the first MDMC pulsed current frequency and the second MDMC pulsed current frequency causes rhythmic contraction of the patient's muscle within the physical manipulation target region at the contraction pulse frequency that is lower than the condition treatment pulse frequency and lower than the tissue treatment pulse frequency.

13. The MDMC electrotherapy system of claim 8, wherein the contraction pulse frequency is less than 5 Hz and the condition treatment pulse frequency are equal to or greater than 10 Hz.

14. The MDMC electrotherapy system of claim 8, wherein the mobile electrode is an electrotherapy glove having an exterior surface including an electrically conductive patient-contact surface for contacting the patient's skin within the physical manipulation target region, during the electrotherapy-assisted physical manipulation treatment.

15. A method of diagnosing a condition of a patient's tissue type using a mixed direct macro-current (MDMC) electrotherapy system comprising:
placing a first electrode and a second electrode in contact with a patient's skin within a physical manipulation target region of a patient's body identified by the patient;
identifying a plurality of tissue types located within the physical manipulation target region;
identifying a plurality of conditions capable of causing one or more of symptoms within the physical manipulation target region, as reported by the patient;
delivering a first MDMC pulsed current frequency having one of a plurality of tissue treatment pulse frequencies corresponding to one of the plurality of tissue types to the patient's skin via the first electrode and a third electrode;
delivering a second MDMC pulsed current frequency having one of a plurality of condition treatment pulse frequencies corresponding to one of the plurality of conditions capable of causing the one or more symptoms via a mobile electrode applied by the practitioner to the patient's skin within the physical manipulation target region via the second electrode and a fourth electrode;
observing rhythmic contraction of the patient's muscles within the area of complaint at a contraction pulse frequency that is lower than at least one of the condition treatment pulse frequency or the tissue treatment pulse frequency; and
diagnosing the condition and tissue type to be treated based on identification of a combination of one of the plurality of condition treatment pulse frequencies and one of the plurality of tissue treatment pulse frequencies that cause the rhythmic contraction.

16. The method of claim 15, wherein the second and fourth electrodes are mobile electrodes.

17. The method of claim 16, wherein the mobile electrodes are on an electrotherapy glove used to provide physical manipulation treatment in addition to electrotherapy treatment with the condition treatment frequency and tissue treatment frequency identified to identify the condition affecting the identified tissue in the physical manipulation target region.

18. The method of claim 15, wherein the first MDMC pulsed current frequency and the second MDMC pulsed current frequency each have a direct macro-current component and an alternating current (AC) frequency component.

19. The method of claim 15, wherein the tissue type to be treated is a muscle.

20. The method of claim 15, wherein the condition to be treated is a muscle spasm.
